(19) United States
(12) Patent Application Publication
(10) Pub. No.: US 2021/0298962 A1
(43) Pub. Date: Sep. 30, 2021

(54) FEMININE HYGIENE ABSORBENT ARTICLE

(71) Applicant: BASF SE, Ludwigshafen am Rhein (DE)

(72) Inventors: Hui Ge, Shanghai (CN); Norbert Herfert, Shanghai (CN); Mark Elliott, Ludwigshafen (DE); Michael Mitchell, Charlotte, NC (US); Le LI, Shanghai (CN); Gao Min Sun, Shanghai (CN)

(21) Appl. No.: 17/263,781

(22) PCT Filed: Jul. 23, 2019
(86) PCT No.: PCT/EP2019/069831

§ 371 Date: Jan. 27, 2021

(30) Foreign Application Priority Data
Aug. 1, 2018 (CN) PCT/CN2018/098042

(51) Int. Cl. A61F 13/472; A61F 13/53; A61L 15/60; C08F 220/06; A61L 15/24

(52) U.S. Cl. CPC A61F 13/472; A61F 13/53; A61L 15/24; C08F 220/06; A61L 15/60

(57) ABSTRACT

Described herein is a feminine hygiene absorbent article, including:
(A) an upper liquid-pervious layer,
(B) a lower liquid-impervious layer,
(C) a fluid-absorbent core between the layer (A) and the layer (B), including 5% to 50% by weight of water-absorbent polymer particles G and not more than 95% by weight of fibrous material, based on the sum of water-absorbent polymer particles G and fibrous material;
(D) an optional acquisition-distribution layer between (A) and (C),
(E) an optional tissue layer disposed immediately above and/or below (C); and
(F) other optional components,
   where the water-absorbent polymer particles G are obtainable by agglomerating a blend of non-surface post-crosslinked fine water-absorbent polymer particles and surface post-crosslinked fine water-absorbent polymer particles, and drying, grinding, sieving and classifying the agglomerated water-absorbent polymer particles.

The present invention relates to a feminine hygiene absorbent article, comprising
(A) an upper liquid-pervious layer,
(B) a lower liquid-impervious layer,
(C) a fluid-absorbent core between the layer (A) and the layer (B), comprising 5% to 50% by weight of water-absorbent polymer particles G and not more than 95% by weight of fibrous material, based on the sum of water-absorbent polymer particles G and fibrous material;
(D) an optional acquisition-distribution layer between (A) and (C),
(E) an optional tissue layer disposed immediately above and/or below (C); and
(F) other optional components,
   wherein the water-absorbent polymer particles G being obtainable by agglomerating a blend of non-surface post-crosslinked fine water-absorbent polymer particles and surface post-crosslinked fine water-absorbent polymer particles drying, grinding, sieving and classification of the agglomerated water-absorbent polymer particles.

Absorbent articles for absorption of proteinaceous or serous body fluids such as menses blood, plasma, vaginal secretions or milk are well known in the art. Typically such absorbent articles comprise feminine hygiene articles such as tampons, sanitary napkins, pantiliners, tampons, and interlabial devices, as well as wound dressings, breast pads or the like. The purpose of such articles is to receive and/or absorb and/or contain and/or retain said body fluids.

Certain absorbent articles such as e.g. sanitary napkins and pantiliners typically include a fluid pervious topsheet as wearer-facing layer, a backsheet as garment-facing layer, which may be fluid impervious and/or may be water vapour and/or gas pervious, and an absorbent core comprised there between. The body fluids are acquired through the topsheet and subsequently stored in the absorbent core. The backsheet providing liquid containment such that absorbed liquid does not leak through the article.

The production of fluid-absorbent articles generally is described in the monograph “Modern Superabsorbent Polymer Technology”, F. L. Buchholz and A. T. Graham, Wiley-VCH, 1998, pages 252 to 258.

Typically, the absorbent core comprises one or more fibrous materials and water-absorbing polymer particles in dispersed form, e.g. typically in particulate form.

The preparation of water-absorbing polymer particles is likewise described in the monograph “Modern Superabsorbent Polymer Technology”, F. L. Buchholz and A. T. Graham, Wiley-VCH, 1998, pages 71 to 103. The water-absorbing polymer particles are also referred to as “fluid-absorbing polymer particles”, “superabsorbent polymers” or “superabsorbents”.

Conventional water-absorbing polymer particles known in the art for use in absorbent articles typically have good absorption and retention characteristics to water and urine; however, there still remains room for improvement for absorption and retention towards proteinaceous or serous body fluids such as typically menses, blood, plasma, vaginal secretions or milk.

Conventional water-absorbing polymer particles often show a slow initial uptake rate for such fluids, which can result in a low final absorption and retention capacity if gel blocking occurs before the water-absorbing polymer particles are fully swollen.

Attempts to increase the absorption and retention capacity of superabsorbent materials for proteinaceous or serous fluids have led for example to chemical modification of these water-absorbing polymer particles, such as by differential crosslinking between surface and bulk of the particle, or treatment with additives to improve wettability with blood by surface treatment.

But it is still a problem to provide feminine hygiene absorbent articles for absorption of proteinaceous or serous body fluids resulting in low rewet. Especially at high loadings the wet feeling is still a problem

Thus, an absorbent article structure is desired exhibiting thinness for comfort combined with high absorbent capacity, while at the same time providing low rewet and fast fluid acquisition.

There is also a need for feminine hygiene absorbent articles with less visibility of the body fluid absorbed, especially menses, blood and improved cleanness for the skin of the wearer.

It is therefore an object of the present invention to provide feminine hygiene absorbent articles comprising water-absorbing polymer particles with improved acquisition of proteinaceous or serous body fluids and retention behavior and improved rewet performance.

It is also an object of the present invention to provide feminine hygiene absorbent articles with less visibility of the absorbed body fluid and improved cleanness of the wearers skin.

The object is achieved by a feminine hygiene absorbent article, comprising
(A) an upper liquid-pervious layer,
(B) a lower liquid-impervious layer,
(C) a fluid-absorbent core between the layer (A) and the layer (B), comprising 5% to 50% by weight of water-absorbent polymer particles G and not more than 95% by weight of fibrous material, based on the sum of water-absorbent polymer particles G and fibrous material;
(D) an optional acquisition-distribution layer between (A) and (C),
(E) an optional tissue layer disposed immediately above and/or below (C); and
(F) other optional components,
   wherein the water-absorbent polymer particles G being obtainable by agglomerating a blend of non-surface post-crosslinked fine water-absorbent polymer particles and surface post-crosslinked fine water-absorbent polymer particles drying, grinding, sieving and classification of the agglomerated water-absorbent polymer particles.

According to the invention for the agglomeration of the fine water-absorbent polymer particles a solution or suspension comprising,
a) 0.04 to 1.2% by weight water-soluble or water-dispersible polymeric binders, based on the water-absorbent polymer particles,
b) 20 to 70% by weight of water based on the water-absorbent polymer particles, and
c) 5 to 20% by weight of a water-miscible organic solvent based on the water-absorbent polymer particles, is used.

According to the invention the fine water-absorbent polymer particles having an average particle diameter of not larger than 300 μm. The fine water-absorbent polymer particles according to the invention preferably having an average particle diameter of at maximum 150 μm, more preferably at maximum 120 μm.

In an embodiment of the invention the ratio of the non-surface post-crosslinked fine water-absorbent polymer particles to the surface post-crosslinked fine water-absorbent polymer particles is preferably at least 2 to 1 by weight.

In a preferred embodiment of the inventive feminine hygiene absorbent article the ratio of the non-surface post-crosslinked fine water-absorbent polymer particles to the surface post-crosslinked fine water-absorbent polymer particles is at least 3 to 1 by weight.

According to the invention it is preferred that the water-absorbent polymer particles G obtained by agglomeration of fine water-absorbent polymer particles having a blood acquisition time of less than 30 s, wherein the blood acquisition time is measured according to the Blood Acquisition Time test method.

It is furthermore preferred according to the invention that the water-absorbent polymer particles G obtained by agglomeration of fine water-absorbent polymer particles having a milk absorption time of less than 15 s, wherein the milk absorption time is measured according to the Milk Absorption Time test method.

According to the invention the fluid absorbent core (C) of the inventive feminine hygiene absorbent article comprising at maximum 25% by weight of water-absorbent polymer particles G and not less than 75% by weight of fibrous material, based on the sum of water-absorbent polymer particles G and fibrous material.

According to another embodiment of the feminine hygiene absorbent article the fluid-absorbent core (C) comprising 5% to 25% by weight of water-absorbent polymer particles G and 75% to 95% by weight of fibrous material, based on the sum of water-absorbent polymer particles G and fibrous material.

The quantity of water-absorbent polymer particles G within the fluid-absorbent core is from 0.1 to 20 g, preferably from 0.15 to 15 g, and from 0.2 to 10 g in light-incontinence products, and from 0.3 g to 5 g e.g. in sanitary napkins, and in the case of adult diapers up to about 50 g. Preferably the amount of water-absorbent polymer particles G in the core of the inventive feminine hygiene absorbent article is from 0.3 g to 5 g, more preferably from of 0.35 g to 2 g, most preferably from 0.4 g to 1 g.

According to the invention the resulting water-absorbent polymer particles G having a bulk density of 0.55 g/ml or lower.

According to the invention it is preferred that the water-absorbent polymer particles G within the fluid-absorbent core having a bulk density of 0.41 g/ml to 0.55 g/ml, more preferably 0.42 g/ml to 0.49 g/ml.

According to the invention the water-absorbent polymer particles G within the fluid-absorbent core having a vortex of less than 30 s.

According to another embodiment of the invention the water-absorbent polymer particles G within the fluid-absorbent core having a vortex of less than 25 s, preferably less than 20 s, more preferably less than 15 s, most preferably less than 10 s.

According to the invention the vortex of water-absorbent polymer particles G is preferably lower than the vortex of the non-agglomerated non-surface post-crosslinked fine water-absorbent polymer particles, surface post-crosslinked fine water-absorbent polymer particles or mixtures thereof used for its production.

According to the invention the water-absorbent polymer particles G within the fluid-absorbent core having CRC of at least 15 g/g, preferably of at least 18 g/g, more preferably of at least 20 g/g.

The water-absorbent polymer particles G therefore possess a high centrifuge retention capacity which impart good liquid distribution when used in hygiene articles. Furthermore, feminine hygiene absorbent articles according to one embodiment of the present invention comprise low absolute amounts of water-absorbent particles while maintaining excellent dryness.

The inventive absorbent article provides an improved liquid acquisition and retention behavior. The acquisition of the proteinaceous or serous body fluids is faster and the rewet at least the same or even lower than for comparable feminine absorbent articles comprising water-absorbent polymer particles known in the art.

According to one further embodiment of the present invention the basis weight of the fluid-absorbent core is of at maximum 450 gsm.

The inventive feminine hygiene absorbent article, e.g. catamenial devices such as a sanitary napkin or pantiliner typically comprising: an upper liquid-pervious layer or top sheet (A), facing the user of the article during use and being liquid pervious in order to allow liquids, particularly body fluids, to pass into the article; a lower liquid-impervious layer or backsheet (B), providing liquid containment such that absorbed liquid does not leak through the article, this backsheet conventionally providing the garment facing surface of the article; and an absorbent core comprised between the top sheet and the backsheet and providing the absorbent capacity of the article to acquire and retain liquid which has entered the article through the topsheet. All absorbent articles of the present invention however have an absorbent core (C), which can be any absorbent means provided in the article and which is capable of absorbing and retaining body fluids, especially proteinaceous or serous body fluids, such as for example menses.

The absorbent article may also include such other features as are known in the art including, but not limited to, re-closable fastening system, lotion, acquisition layers, distribution layers, wetness indicators, sensors, elasticized waist bands and other similar additional elastic elements and the like, belts and the like, waist cap features, containment and aesthetic characteristics and combinations thereof.

It is furthermore preferred that the basis weight of the fluid-absorbent core preferably at the insult zone is of at maximum 1000 gsm, more preferably of at maximum 750 gsm, preferentially of at maximum 600 gsm, particularly preferred of at maximum 450 gsm

Usually water-absorbent polymer particles are produced by a process, comprising the steps forming water-absorbent polymer particles by polymerizing a monomer solution, comprising
a) at least one ethylenically unsaturated monomer which bears acid groups and may be at least partly neutralized,
b) optionally one or more crosslinker,
c) at least one initiator
d) optionally one or more ethylenically unsaturated monomers copolymerizable with the monomers mentioned under a),
e) optionally one or more water-soluble polymers, and
f) water,
   optionally coating of water-absorbent polymer particles with at least one surface-post-crosslinker and thermal surface-postcrosslinking of the coated water-absorbent polymer particles drying, optionally grinding, sieving and classification of the water-absorbent polymer particles.

Fine water-absorbent polymer particles are usually removed from the superabsorbent production.

According to the present invention the fine water-absorbent polymer particles are collected before and/or after surface-post-crosslinking and agglomerated.

According to the invention the ratio of non-surface post-crosslinked fine water-absorbent polymer particles to surface post-crosslinked fine water-absorbent polymer particles is at least 1:1 by weight, preferably 2:1 by weight, more preferably 3:1 by weight for agglomeration.

According to the invention the blend of fine water-absorbent polymer particles to be agglomerated comprises at least 50% by weight of non-surface post-crosslinked fine water-absorbent polymer particles and at maximum 50% by weight of surface post-crosslinked fine water-absorbent polymer particles.

Preferably the blend comprises at least 60% by weight of non-surface post-crosslinked fine water-absorbent polymer particles and at maximum 40% by weight of surface post-crosslinked fine water-absorbent polymer particles, more preferably the blend comprises at least 70% by weight of non-surface post-crosslinked fine water-absorbent polymer particles and at maximum 30% by weight of surface post-crosslinked fine water-absorbent polymer particles.

For agglomeration a solution or suspension comprising water, water-miscible organic solvents, water-soluble and/or water-dispersible polymeric binders are sprayed onto the fine water-absorbent polymer particles. The spraying with the solution or suspension can, for example, be carried out in mixers having moving mixing implements, such as screw mixers, paddle mixers, disk mixers, plowshare mixers and shovel mixers. Useful mixers include for example Lodige® mixers, Bepex® mixers, Nauta® mixers, Processall® mixers and Schugi® mixers. Vertical mixers are preferred. Fluidized bed apparatuses are particularly preferred.

### DETAILED DESCRIPTION OF THE INVENTION

### A. Definitions

As used herein, the term “fluid-absorbent article” or “feminine hygiene absorbent article” refers to any three-dimensional solid material being able to acquire and store especially proteinaceous or serous body fluids such as menses blood, plasma, vaginal secretions or milk discharged from the body. Preferred fluid-absorbent articles are disposable fluid-absorbent articles that are designed to be worn in contact with the body of a user. Typically, such absorbent articles comprise fFeminine hygiene absorbent articles such as breast pads, sanitary napkins, tampons, pantiliner and interlabial devices, as well as wound dressings or other articles useful for absorbing body fluids, such as for low or moderate adult incontinence, as e.g. incontinence pads and incontinence briefs for adults. Suitable feminine hygiene absorbent articles including fluid-absorbent compositions comprising fibrous materials and water-absorbent polymer particles to form fibrous webs or matrices for the substrates, layers, sheets and/or the fluid-absorbent core.

As used herein, the term “fluid-absorbent composition” refers to a component of the fluid-absorbent article which is primarily responsible for the fluid handling of the fluid-absorbent article including acquisition, transport, distribution and storage of body fluids.

As used herein, the term “fluid-absorbent core”, “absorbent core” refers to a fluid-absorbent composition comprising at least one layer of water-absorbent polymer particles and fibrous material, nonwoven material and tissue material tissue material and optionally adhesive. The fluid-absorbent core is primarily responsible for the fluid handling of the fluid-absorbent article including acquisition, transport, distribution and storage of body fluids.

As used herein, the term “layer” refers to a fluid-absorbent composition whose primary dimension is along its length and width. Thus a layer can comprise laminates, composites, combinations of several sheets or webs of different materials.

As used herein the term “x-dimension” refers to the length, and the term “y-dimension” refers to the width of the fluid-absorbent composition, layer, core or article. Generally, the term “x-y-dimension” refers to the plane, orthogonal to the height or thickness of the fluid-absorbent composition, layer, core or article.

As used herein the term “z-dimension” refers to the dimension orthogonal to the length and width of the fluid absorbent composition, layer, core or article. Generally, the term “z-dimension” refers to the height of the fluid-absorbent composition, layer, core or article.

As used herein, the term “basis weight” indicates the weight of the fluid-absorbent core per square meter and it includes the chassis of the fluid-absorbent article. The basis weight is determined at discrete regions of the fluid-absorbent core: the front overall average is the basis weight of the fluid-absorbent core 5.5 cm forward of the center of the core to the front distal edge of the core; the insult zone is the basis weight of the fluid-absorbent core 5.5 cm forward and 0.5 cm backwards of the center of the core; the back overall average is the basis weight of the fluid-absorbent core 0.5 cm backward of the center of the core to the rear distal edge of the core.

Further, it should be understood, that the term “upper” refers to fluid-absorbent composition which are nearer to the wearer of the fluid-absorbent article. Generally, the topsheet is the nearest composition to the wearer of the fluid-absorbent article, hereinafter described as “upper liquid-pervious layer”. Contrarily, the term “lower” refers to fluid-absorbent compositions which are away from the wearer of the fluid-absorbent article. Generally, the backsheet is the component which is furthermost away from the wearer of the fluid-absorbent article, hereinafter described as “lower liquid-impervious layer”.

As used herein, the term “liquid-pervious” refers to a substrate, layer or a laminate thus permitting liquids, i.e. body fluids such as menses and/or vaginal fluids to readily penetrate through its thickness.

As used herein, the term “liquid-impervious” refers to a substrate, layer or a laminate that does not allow body fluids to pass through in a direction generally perpendicular to the plane of the layer at the point of liquid contact under ordinary use conditions.

As used herein, the term “chassis” refers to fluid-absorbent material comprising the upper liquid-pervious layer and the lower liquid-impervious layer, elastication and closure systems for the absorbent article.

As used herein, the term “hydrophilic” refers to the wettability of fibers by water deposited on these fibers. The term “hydrophilic” is defined by the contact angle and surface tension of the body fluids. According to the definition of Robert F. Gould in the 1964 American Chemical Society publication “Contact angle, wettability and adhesion”, a fiber is referred to as hydrophilic, when the contact angle between the liquid and the fiber, especially the fiber sur-face, is less than 90° or when the liquid tends to spread spontaneously on the same surface.

Contrarily, term “hydrophobic” refers to fibers showing a contact angle of greater than 90° or no spontaneously spreading of the liquid across the surface of the fiber.

As used herein, the term “body fluids” refers to any fluid produced and discharged by human or animal body, such as urine, menstrual fluids, faeces, vaginal secretions, especially proteinaceous or serous body fluids and the like.

As used herein, the term “breathable” refers to a substrate, layer, film or a laminate that allows vapour to escape from the fluid-absorbent article, while still preventing fluids from leakage. Breathable substrates, layers, films or laminates may be porous polymeric films, nonwoven laminates from spunbond and melt-blown layers, laminates from porous polymeric films and nonwovens.

As used herein, the term “longitudinal” refers to a direction running perpendicular from a waist edge to an opposing waist edge of the fluid-absorbent article.

As used herein the term “fine water-absorbent polymer particles” refers to water-absorbent polymer particles having an average particle diameter of not larger than 300 μm. The fine water-absorbent polymer particles preferably having an average particle diameter of at maximum 150 μm, more preferably at maximum 120 μm.

### B. Water-Absorbent Polymer Particles

Water-absorbent polymer particles, e. g. the fine water-absorbent polymer particles, are generally prepared by a process, comprising the steps forming water-absorbent polymer particles by polymerizing a monomer solution, comprising
a) at least one ethylenically unsaturated monomer which bears acid groups and may be at least partly neutralized,
b) optionally one or more crosslinker,
c) at least one initiator,
d) optionally one or more ethylenically unsaturated monomers copolymerizable with the monomers mentioned under a),
e) optionally one or more water-soluble polymers, and
f) water,
   optionally coating of water-absorbent polymer particles with at least one surface-post-crosslinker and thermal surface-post-crosslinking of the coated water-absorbent polymer particles.

The water-absorbent polymer particles are typically insoluble but swellable in water.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids such as acrylic acid, methacrylic acid, maleic acid, and itaconic acid. Particularly preferred monomers are acrylic acid and methacrylic acid. Very particular preference is given to acrylic acid.

Further suitable monomers a) are, for example, ethylenically unsaturated sulfonic acids such as vinylsulfonic acid, styrenesulfonic acid and 2-acrylamido-2-methylpropanesulfonic acid (AMPS).

Impurities may have a strong impact on the polymerization. Preference is given to especially purified monomers a). Useful purification methods are disclosed in WO 2002/055469 A1, WO 2003/078378 A1 and WO 2004/035514 A1. A suitable monomer a) is according to WO 2004/035514 A1 purified acrylic acid having 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

Polymerized diacrylic acid is a source for residual monomers due to thermal decomposition. If the temperatures during the process are low, the concentration of diacrylic acid is no more critical and acrylic acids having higher concentrations of diacrylic acid, i.e. 500 to 10,000 ppm, can be used for the inventive process.

The content of acrylic acid and/or salts thereof in the total amount of monomers a) is preferably at least 50 mol %, more preferably at least 90 mol %, most preferably at least 95 mol %.

The acid groups of the monomers a) are typically partly neutralized in the range of 0 to 100 mol %, preferably to an extent of from 25 to 85 mol %, preferentially to an extent of from 50 to 80 mol %, more preferably from 60 to 75 mol %, for which the customary neutralizing agents can be used, preferably alkali metal hydroxides, alkali metal oxides, alkali metal carbonates or alkali metal hydrogen carbonates, and mixtures thereof. Instead of alkali metal salts, it is also possible to use ammonia or organic amines, for example, triethanolamine. It is also possible to use oxides, carbonates, hydrogencarbonates and hydroxides of magnesium, calcium, strontium, zinc or aluminum as powders, slurries or solutions and mixtures of any of the above neutralization agents. Example for a mixture is a solution of sodiumaluminate. Sodium and potassium are particularly preferred as alkali metals, but very particular preference is given to sodium hydroxide, sodium carbonate or sodium hydrogen carbonate, and mixtures thereof. Typically, the neutralization is achieved by mixing in the neutralizing agent as an aqueous solution, as a melt or preferably also as a solid. For example, sodium hydroxide with water content significantly below 50% by weight may be present as a waxy material having a melting point above 23° C. In this case, metered addition as piece material or melt at elevated temperature is possible.

Optionally, it is possible to add to the monomer solution, or to starting materials thereof, one or more chelating agents for masking metal ions, for example iron, for the purpose of stabilization. Suitable chelating agents are, for example, alkali metal citrates, citric acid, alkali metal tartrates, alkali metal lactates and glycolates, pentasodium triphosphate, ethylenediamine tetraacetate, nitrilotriacetic acid, and all chelating agents known under the Trilon® name, for example Trilon® C (pentasodium diethylenetriaminepentaacetate), Trilon® D (trisodium (hydroxyethyl)-ethylenediaminetriacetate), and Trilon® M (methylglycinediacetic acid) and Cublen®.

The monomers a) comprise typically polymerization inhibitors, preferably hydroquinone monoethers, as inhibitor for storage.

The monomer solution comprises preferably up to 250 ppm by weight, more preferably not more than 130 ppm by weight, most preferably not more than 70 ppm by weight, preferably not less than 10 ppm by weight, more preferably not less than 30 ppm by weight and especially about 50 ppm by weight of hydroquinone monoether, based in each case on acrylic acid, with acrylic acid salts being counted as acrylic acid. For example, the monomer solution can be prepared using acrylic acid having appropriate hydroquinone monoether content. The hydroquinone monoethers may, however, also be removed from the monomer solution by absorption, for example on activated carbon.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized by a free-radical mechanism into the polymer chain and functional groups which can form covalent bonds with the acid groups of monomer a). In addition, polyvalent metal ions which can form coordinate bond with at least two acid groups of monomer a) are also suitable crosslinkers b).

The crosslinkers b) are preferably compounds having at least two free-radically polymerizable groups which can be polymerized by a free-radical mechanism into the polymer network. Suitable crosslinkers b) are, for example, ethylene glycol dimethacrylate, diethylene glycol diacrylate, polyethylene glycol diacrylate, allyl methacrylate, trime-thylolpropane triacrylate, triallylamine, tetraallylammonium chloride, tetraallyloxyethane, as described in EP 0530438 A1, di- and triacrylates, as described in EP 0547847 A1, EP 0559476 A1, EP 0632068 A1, WO 93/21237 A1, WO 2003/104299 A1, WO 2003/104300 A1, WO 2003/104301 A1 and in DE 10331450 A1, mixed acrylates which, as well as acrylate groups, comprise further ethylenically unsaturated groups, as described in DE 10331456 A1 and DE 10355401 A1, or crosslinker mixtures, as described, for example, in DE 19543368 A1, DE 19646484 A1, WO 90/15830 A1 and WO 2002/32962 A2.

Suitable crosslinkers b) are in particular pentaerythritol triallyl ether, tetraallyloxyethane, polyethyleneglycole dial-lylethers (based on polyethylene glycole having a molecular weight between 400 and 20000 g/mol), N,N′-methylenebisacrylamide, 15-tuply ethoxylated trimethylolpropane, polyethylene glycol diacrylate, trimethylolpro-pane triacrylate and triallylamine.

Very particularly preferred crosslinkers b) are the polyethoxylated and/or -propoxylated glycerols which have been esterified with acrylic acid or methacrylic acid to give di- or triacrylates, as described, for example in WO 2003/104301 A1. Di- and/or triacrylates of 3- to 18-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol and especially the triacrylate of 3-tuply ethoxylated glycerol.

The amount of crosslinker b) is preferably from 0.0001 to 0.6% by weight, more preferably from 0.001 to 0.2% by weight, most preferably from 0.01 to 0.06% by weight, based in each case on monomer a). On increasing the amount of crosslinker b) the centrifuge retention capacity (CRC) decreases and the absorption under a pressure of 21.0 g/cm² (AUL) passes through a maximum.

The initiators c) used may be all compounds which disintegrate into free radicals under the polymerization conditions, for example peroxides, hydroperoxides, hydrogen peroxide, persulfates, azo compounds and redox initiators. Preference is given to the use of water-soluble initiators. In some cases, it is advantageous to use mixtures of various initiators, for example mixtures of hydrogen peroxide and sodium or potassium peroxodisulfate. Mixtures of hydrogen peroxide and sodium peroxodisulfate can be used in any proportion.

Particularly preferred initiators c) are azo initiators such as 2,2-azobis[2-(2-imidazolin-2-yl)propane]digydrocloride and 2,2′-azobis[2-(5-methyl-2-imidazolin-2-yl)propane]dihydrochloride, 2,2′-azobis(2-amidinopropane) dihy-drochloride, 4,4′-azobis(4-cyanopentanoic acid), 4,4′-azobis(4-cyanopentanoic acid) sodium salt, 2,2′-azobis[2-methyl-N-(2-hydroxyethyl)propionamide], and photoinitiators such as 2-hydroxy-2-methylpropiophenone and 1-[4-(2-hydroxyethoxy)phenyl]-2-hydroxy-2-methyl-1-propan-1-one, redox initiators such as sodium persulfate/hydroxymethylsulfinic acid, ammonium peroxodisulfate/hydroxymethylsulfinic acid, hydrogen peroxide/hydroxymethylsulfinic acid, sodium persulfate/ascorbic acid, ammonium peroxodisulfate/ascorbic acid and hydrogen peroxide/ascorbic acid, photoinitiators such as 1-[4-(2-hydroxyethoxy)phenyl]-2-hydroxy-2-methyl-1-propan-1-one, and mixtures thereof. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany). Of course it is also possible within the scope of the present invention to use the purified salts or acids of 2-hydroxy-2-sulfinatoacetic acid and 2-hydroxy-2-sulfonatoacetic acid—the latter being available as sodium salt un-der the trade name Blancolen® (Brüggemann Chemicals; Heilbronn; Germany).

The initiators are used in customary amounts, for example in amounts of from 0.001 to 5% by weight, preferably from 0.01 to 2% by weight, most preferably from 0.05 to 0.5% by weight, based on the monomers a).

Examples of ethylenically unsaturated monomers d) which are copolymerizable with the monomers a) are acrylamide, methacrylamide, hydroxyethyl acrylate, hydroxyethyl methacrylate, dimethylaminoethyl acrylate, dimethyl-aminoethyl methacrylate, dimethylaminopropyl acrylate and diethylaminopropyl methacrylate.

Useful water-soluble polymers e) include polyvinyl alcohol, modified polyvinyl alcohol comprising acidic side groups for example Poval® K (Kuraray Europe GmbH; Frankfurt; Germany), polyvinylpyrrolidone, starch, starch derivatives, modified cellulose such as methylcellulose, carboxymethylcellulose or hydroxyethylcellulose, gelatin, polyglycols or polyacrylic acids, polyesters and polyamides, polylactic acid, polyglycolic acid, co-polylactic-polyglycolic acid, polyvinylamine, polyallylamine, water soluble copolymers of acrylic acid and maleic acid available as Sokalan® (BASF SE; Ludwigshafen; Germany), preferably starch, starch derivatives and modified cellulose.

For optimal action, the preferred polymerization inhibitors require dissolved oxygen. Therefore, the monomer solution can be freed of dissolved oxygen before the polymerization by inertization, i.e. flowing through with an inert gas, preferably nitrogen. It is also possible to reduce the concentration of dissolved oxygen by adding a reducing agent. The oxygen content of the monomer solution is preferably lowered before the polymerization to less than 1 ppm by weight, more preferably to less than 0.5 ppm by weight.

The water content of the monomer solution is preferably less than 65% by weight, preferentially less than 62% by weight, more preferably less than 60% by weight, most preferably less than 58% by weight.

The monomer solution has, at 20° C., a dynamic viscosity of preferably from 0.002 to 0.02 Pa·s, more preferably from 0.004 to 0.015 Pa·s, most preferably from 0.005 to 0.01 Pa·s.

The monomer solution has, at 20° C., a density of preferably from 1 to 1.3 g/cm³, more preferably from 1.05 to 1.25 g/cm³, most preferably from 1.1 to 1.2 g/cm³.

The monomer solution has, at 20° C., a surface tension of from 0.02 to 0.06 N/m, more preferably from 0.03 to 0.05 N/m, most preferably from 0.035 to 0.045 N/m.

### Polymerization

The monomer solution is polymerized. Suitable reactors are, for example, kneading reactors or belt reactors. In the kneader, the polymer gel formed in the polymerization of an aqueous monomer solution or suspension is comminuted continuously by, for example, contra-rotatory stirrer shafts, as described in WO 2001/038402 A1. Polymerization on the belt is described, for example, in DE 3825366 A1 and U.S. Pat. No. 6,241,928. Polymerization in a belt reactor forms a polymer gel which has to be comminuted in a further process step, for example in an extruder or kneader

To improve the drying properties, the comminuted polymer gel obtained by means of a kneader can additionally be extruded.

The polymer gel is then preferably dried with a belt dryer until the residual moisture content is preferably from 0.5 to 15% by weight, more preferably from 1 to 10% by weight, most preferably from 2 to 8% by weight, the residual moisture content being determined by the EDANA recommended test method No. WSP 230.2-05 “Moisture Content”. In the case of too high a residual moisture content, the dried polymer gel has too low a glass transition temperature Tg and can be processed further only with difficulty. In the case of too low a residual moisture content, the dried polymer gel is too brittle and, in the subsequent comminution steps, undesirably large amounts of polymer particles with an excessively low particle size are obtained (fines). The solids content of the gel before the drying is preferably from 25 to 90% by weight, more preferably from 35 to 70% by weight, most preferably from 40 to 60% by weight. Optionally, it is, however, also possible to use a fluidized bed dryer or a paddle dryer for the drying operation. Thereafter, the dried polymer gel is ground and classified, and the apparatus used for grinding may typically be single- or multistage roll mills, preferably two- or three-stage roll mills, pin mills, hammer mills or vibratory mills.

Alternatively, the water-absorbent polymer particles are produced by polymerizing droplets of the monomer in a surrounding heated gas phase, for example by using a system described in WO 2008/040715 A2, WO 2008/052971 A1, WO 2008/069639 A1 and WO 2008/086976 A1.

The mean particle size or also referred to as average particle diameter of the water-absorbentpolymer particles removed as the product fraction is preferably above 120 μm, more preferably above 150 μm, most preferably from 250 to 600 μm and very particularly preferable from 300 to 500 μm. The mean particle size of the product fraction may be determined by means of EDANA (European Disposables and Nonwovens Association) recommended test method No. WSP 220.3 (11) “Particle Size Distribution”, where the proportions by mass of the screen fractions are plotted in cumulated form and the mean particle size is determined graphically. The mean particle size or average particle diameter here is the value of the mesh size which gives rise to a cumulative 50% by weight.

The fine water-absorbent polymer particles are removed as they are reducing the product performance in superabsorbent products.

According to the present invention the fine water-absorbent polymer particles, which are removed at this process step or in later process steps, for example after the surface post-crosslinking or another coating step are collected and agglomerated. So that they result in water-absorbent polymer particles G.

The later process steps, for example the surface post-crosslinking or coating steps are generally described in the following:

### Surface-Post-Crosslinking

Surface-post-crosslinkers are compounds which comprise groups which can form at least two covalent bonds with the carboxylate groups of the polymer particles. Suitable compounds are, for example, polyfunctional amines, polyfunctional amidoamines, polyfunctional epoxides, as described in EP 0083022 A2, EP 0543303 A1 and EP 0937736 A2, di- or polyfunctional alcohols as described in DE 3314019 A1, DE 3523617 A1 and EP 0450922 A2, or β-hydroxyalkylamides, as described in DE 10204938 A1 and U.S. Pat. No. 6,239,230. Also ethylene oxide, aziridine, glycidol, oxetane and its derivatives may be used.

Polyvinylamine, polyamidoamines and polyvinylalcohols are examples of multifunctional polymeric surface-post-crosslinkers.

In addition, DE 4020780 C1 describes alkylene carbonates, DE 19807502 A1 describes 1,3 oxazolidin-2-one and its derivatives such as 2-hydroxyethyl-1,3-oxazolidin-2-one, DE 19807 992 C1 describes bis- and poly-1,3-oxazolidin-2-ones, EP 0999238 A1 describes bis- and poly-1,3-oxazolidines, DE 19854573 A1 describes 2 oxotetrahydro-1,3-oxazine and its derivatives, DE 19854574 A1 describes N-acyl-1,3 oxazolidin-2-ones, DE 10204937 A1 describes cyclic ureas, DE 10334584 A1 describes bicyclic amide acetals, EP 1199327 A2 describes oxetanes and cyclic ureas, and WO 2003/31482 A1 describes morpholine-2,3-dione and its derivatives, as suitable surface-post-crosslinkers.

In addition, it is also possible to use surface-post-crosslinkers which comprise additional polymerizable ethylenically unsaturated groups e.g 1,3,2-dioxathiolane, as e.g. described in DE 3713601 A1.

The at least one surface-post-crosslinker is selected from alkylene carbonates, 1,3 oxazolidin-2-ones, bis- and poly-1,3-oxazolidin-2-ones, bis- and poly-1,3-oxazolidines, 2 oxotetrahydro-1,3-oxazines, N-acyl-1,3 oxazolidin-2-ones, cyclic ureas, bicyclic amide acetals, oxetanes, and morpholine-2,3-diones. Suitable surface-post-crosslinkers are ethylene carbonate, 3-methyl-1,3-oxazolidin-2-one, 3-methyl-3-oxethanmethanol, 1,3-oxazolidin-2-one, 3-(2-hydroxyethyl)-1,3-oxazolidin-2-one, 1,3-dioxan-2-one or a mixture thereof.

It is also possible to use any suitable mixture of surface-post-crosslinkers. It is particularly favorable to use mixtures of 1,3-dioxolan-2-on (ethylene carbonate) and 1,3 oxazolidin-2-ones. Such mixtures are obtainable by mixing and partly reacting of 1,3-dioxolan-2-on (ethylene carbonate) with the corresponding 2-amino-alcohol (e.g. 2-aminoethanol) and may comprise ethylene glycol from the reaction.

It is preferred that at least one alkylene carbonate is used as surface-post-crosslinker. Suitable alkylene carbonates are 1,3-dioxolan-2-on (ethylene carbonate), 4-methyl-1,3-dioxolan-2-on (propylene carbonate), 4,5-dimethyl-1,3-dioxolan-2-on, 4,4-dimethyl-1,3-dioxolan-2-on, 4-ethyl-1,3-dioxolan-2-on, 4-hydroxymethyl-1,3-dioxolan-2-on (glycerol carbonate), 1,3-dioxane-2-on (trimethylene carbonate), 4-methyl-1,3-dioxane-2-on, 4,6-dimethyl-1,3-dioxane-2-on and 1,3-dioxepan-2-on, preferably 1,3-dioxolan-2-on (ethylene carbonate) and 1,3-dioxane-2-on (trimethylene carbonate), most preferably, 3-dioxolan-2-on (ethylene carbonate).

The amount of surface-post-crosslinker is preferably from 0.1 to 10% by weight, more preferably from 0.5 to 7.5% by weight, most preferably from 1 to 5% by weight, based in each case on the polymer.

The content of residual monomers in the water-absorbent polymer particles prior to the coating with the surface-post-crosslinker is in the range from 0.03 to 15% by weight, preferably from 0.05 to 12% by weight, more preferably from 0.1 to 10% by weight, even more preferably from 0.15 to 7.5% by weight, most preferably from 0.2 to 5% by weight, even most preferably from 0.25 to 2.5% by weight.

The moisture content of the water-absorbent polymer particles prior to the thermal surface-post-crosslinking is preferably from 1 to 20% by weight, more preferably from 2 to 15% by weight, most preferably from 3 to 10% by weight.

Polyvalent cations can be applied to the particle surface in addition to the surface-post-crosslinkers before, during or after the thermal surface-post-crosslinking.

The polyvalent cations usable in the process according to the invention are, for example, divalent cations such as the cations of zinc, magnesium, calcium, iron and strontium, trivalent cations such as the cations of aluminum, iron, chromium, rare earths and manganese, tetravalent cations such as the cations of titanium and zirconium, and mixtures thereof. Possible counterions are chloride, bromide, sulfate, hydrogensulfate, methanesulfate, carbonate, hydrogencarbonate, nitrate, hydroxide, phosphate, hydrogenphosphate, dihydrogenphosphate, glycophosphate and carboxylate, such as acetate, glycolate, tartrate, formate, propionate, 3-hydroxypropionate, lactamide and lactate, and mixtures thereof. Aluminum sulfate, aluminum acetate, and aluminum lactate are preferred. Aluminum lactate is more preferred. Using the inventive process in combination with the use of aluminum lactate, water-absorbent polymer particles having an extremely high total liquid uptake at lower centrifuge retention capacities (CRC) can be prepared.

Apart from metal salts, it is also possible to use polyamines and/or polymeric amines as polyvalent cations. A single metal salt can be used as well as any mixture of the metal salts and/or the polyamines above.

Preferred polyvalent cations and corresponding anions are disclosed in WO 2012/045705 A1 and are expressly incorporated herein by reference. Preferred polyvinylamines are disclosed in WO 2004/024816 A1 and are expressly incorporated herein by reference.

The amount of polyvalent cation used is, for example, from 0.001 to 1.5% by weight, preferably from 0.005 to 1% by weight, more preferably from 0.02 to 0.8% by weight, based in each case on the polymer.

The addition of the polyvalent metal cation can take place prior, after, or co-currently with the surface-post-crosslinking. Depending on the formulation and operating conditions employed it is possible to obtain a homogeneous surface coating and distribution of the polyvalent cation or an inhomogeneous typically spotty coating. Both types of coatings and any mixes between them are useful within the scope of the present invention.

The surface-post-crosslinking is typically performed in such a way that a solution of the surface-postcrosslinker is sprayed onto the hydrogel or the dry polymer particles. After the spraying, the polymer particles coated with the surface-post-crosslinker are dried thermally and cooled.

The spraying of a solution of the surface-post-crosslinker is preferably performed in mixers with moving mixing tools, such as screw mixers, disk mixers and paddle mixers. Suitable mixers are, for example, vertical Schugi Flexomix® mixers (Hosokawa Micron BV; Doetinchem; the Netherlands), Turbolizers® mixers (Hosokawa Micron BV; Doetinchem; the Netherlands), horizontal Pflugschar® plowshare mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; US) and Ruberg continuous flow mixers (Gebrüder Ruberg GmbH & Co KG, Nieheim, Germany). Ruberg continuous flow mixers and horizontal Pflugschar® plow-share mixers are preferred. The surface-postcrosslinker solution can also be sprayed into a fluidized bed.

The solution of the surface-post-crosslinker can also be sprayed on the water-absorbent polymer particles during the thermal posttreatment. In such case the surface-post-crosslinker can be added as one portion or in several portions along the axis of thermal posttreatment mixer. In one embodiment it is preferred to add the surface-post-crosslinker at the end of the thermal posttreatment step. As a particular advantage of adding the solution of the surface-post-crosslinker during the thermal posttreatment step it may be possible to eliminate or reduce the I technical effort for a separate surface-post-crosslinker addition mixer.

The surface-post-crosslinkers are typically used as an aqueous solution. The addition of non-aqueous solvent can be used to improve surface wetting and to adjust the penetration depth of the surface-post-crosslinker into the polymer particles.

The thermal surface-post-crosslinking is preferably carried out in contact dryers, more preferably paddle dryers, most preferably disk dryers. Suitable driers are, for example, Ho-sokawa Bepex® horizontal paddle driers (Ho-sokawa Micron GmbH; Leingarten; Germany), Hosokawa Bepex® disk driers (Hosokawa Micron GmbH; Leingarten; Germany), HoloFlite® dryers (Metso Minerals Industries Inc.; Danville; U.S.A.) and Nara paddle driers (NARA Machinery Europe; Frechen; Germany). Moreover, it is also possible to use fluidized bed dryers. In the latter case the reaction times may be shorter compared to other embodiments.

When a horizontal dryer is used then it is often advantageous to set the dryer up with an inclined angle of a few degrees vs. the earth surface in order to impart proper product flow through the dryer. The angle can be fixed or may be adjustable and is typically between 0 to 10 degrees, preferably 1 to 6 degrees, most preferably 2 to 4 degrees.

A contact dryer can be used that has two different heating zones in one apparatus. For example, Nara paddle driers are available with just one heated zone or alternatively with two heated zones. The advantage of using a two or more heated zone dryer is that different phases of the thermal post-treatment and/or of the post-surface-crosslinking can be combined.

It is possible to use a contact dryer with a hot first heating zone which is followed by a temperature holding zone in the same dryer. This set up allows a quick rise of the product temperature and evaporation of surplus liquid in the first heating zone, whereas the rest of the dryer is just holding the product temperature stable to complete the reaction.

It is also possible to use a contact dryer with a warm first heating zone which is then followed by a hot heating zone. In the first warm zone the thermal post-treatment is affected or completed whereas the surface-post-crosslinking takes place in the subsequential hot zone.

Typically, a paddle heater with just one temperature zone is employed.

A person skilled in the art will depending on the desired finished product properties and the available base polymer qualities from the polymerization step choose any one of these set ups.

The thermal surface-post-crosslinking can be effected in the mixer itself, by heating the jacket, blowing in warm air or steam. Equally suitable is a downstream dryer, for example a shelf dryer, a rotary tube oven or a heatable screw. It is particularly advantageous to mix and dry in a fluidized bed dryer.

Preferred thermal surface-post-crosslinking temperatures are usually in the range of 100-195° C., mostly in the range of 100 to 180° C., preferably from 120 to 170° C., more preferably from 130 to 165° C., most preferably from 140 to 160° C. The preferred residence time at this temperature in the reaction mixer or dryer is preferably at least 5 minutes, more preferably at least 20 minutes, most preferably at least 40 minutes, and typically at most 120 minutes.

It is preferable to cool the polymer particles after thermal surface-post-crosslinking. The cooling is preferably carried out in contact coolers, more preferably paddle coolers, most preferably disk coolers. Suitable coolers are, for ex-ample, Hosokawa Bepex® horizontal paddle coolers (Hosokawa Micron GmbH; Leingarten; Germany), Hosokawa Bepex® disk coolers (Hosokawa Micron GmbH; Leingarten; Germany), Holo-Flite® coolers (Metso Minerals Industries Inc.; Danville; U.S.A.) and Nara paddle coolers (NARA Machinery Europe; Frechen; Germany). Moreover, it is also possible to use fluidized bed coolers.

In the cooler the polymer particles are cooled to temperatures in the range from 20 to 150° C., preferably from 40 to 120° C., more preferably from 60 to 100° C., most preferably from 70 to 90° C. Cooling using warm water is preferred, especially when contact coolers are used.

According to the described production process fine water-absorbent polymer particles are typically removed also after post-crosslinking and collected according to the present invention and agglomerated. For agglomeration the fine water-absorbent particles are preferably blended with fine water-absorbent polymer particles collected in at least one different step of the production process.

### Coating

To further improve the properties, the water-absorbent polymer particles can be coated and/or optionally moistened. The internal fluidized bed, the external fluidized bed and/or the external mixer used for the thermal posttreatment and/or a separate coater (mixer) can be used for coating of the water-absorbent polymer particles. Further, the cooler and/or a separate coater (mixer) can be used for coating/moistening of the surface-post-crosslinked water-absorbent polymer particles. Suitable coatings for controlling the acquisition behavior and improving the permeability (SFC or GBP) are, for example, inorganic inert substances, such as water-insoluble metal salts, organic polymers, cationic polymers, anionic polymers and polyvalent metal cations. Suitable coatings for improving the color stability are, for example reducing agents, chelating agents and anti-oxidants. Suitable coatings for dust binding are, for example, polyols. Suitable coatings against the undesired caking tendency of the polymer particles are, for ex-ample, fumed silica, such as Aerosil® 200, and surfactants, such as Span® 20 and Plantacare® 818 UP. Preferred coatings are aluminum dihydroxy monoacetate, aluminum sulfate, aluminum lactate, aluminum 3-hydroxypropionate, zirconium acetate, citric acid or its water-soluble salts, di- and mono-phosphoric acid or their water soluble salts, Blancolen®, Brüggolite® FF7, Cublen®, Span® 20 and Plantacare® 818 UP.

If salts of the above acids are used instead of the free acids then the preferred salts are alkali-metal, earth alkali metal, aluminum, zirconium, titanium, zinc and ammonium salts.

Under the trade name Cublen® (Zschimmer & Schwarz Mohsdorf GmbH & Co KG; Burgstadt; Germany) the following acids and/or their alkali metal salts (preferably Na and K-salts) are available and may be used within the scope of the present invention for example to impart color-stability to the finished product:

1-Hydroxyethane-1,1-diphosphonic acid, Amino-tris(methylene phosphonic acid), Ethylenediamine-tetra(methylene phosphonic acid), Diethylenetriamine-penta(methylene phosphonic acid), Hexamethylene diamine-tetra(methylenephosphonic acid), Hydroxyethylamino-di(methylene phosphonic acid), 2-Phosphonobutane-1,2,4-tricarboxylic acid, Bis(hexamethylenetriamine penta(methylene phosphonic acid).

Most preferably 1-Hydroxyethane-1,1-diphosphonic acid or its salts with sodium, potassium, or ammonium are employed. Any mixture of the above Cublenes® can be used.

Alternatively, any of the chelating agents described before for use in the polymerization can be coated onto the finished product.

Suitable inorganic inert substances are silicates such as montmorillonite, kaolinite and talc, zeolites, activated carbons, polysilicic acids, magnesium carbonate, calcium carbonate, calcium phosphate, aluminum phosphate, barium sulfate, aluminum oxide, titanium dioxide and iron(II) oxide. Preference is given to using polysilicic acids, which are divided between precipitated silicas and fumed silicas according to their mode of preparation. The two variants are commercially available under the names Silica FK, Sipernat®, Wessalon® (precipitated silicas) and Aerosil® (fumed silicas) respectively. The inorganic inert substances may be used as dispersion in an aqueous or water-miscible dispersant or in substance.

When the water-absorbent polymer particles are coated with inorganic inert substances, the amount of inorganic inert substances used, based on the water-absorbent polymer particles, is preferably from 0.05 to 5% by weight, more preferably from 0.1 to 1.5% by weight, most preferably from 0.3 to 1% by weight.

Suitable organic polymers are polyalkyl methacrylates or thermoplastics such as polyvinyl chloride, waxes based on polyethylene, polypropylene, polyamides or polytetrafluoro-ethylene. Other examples are styrene-isoprene-styrene block-copolymers or styrene-butadiene-styrene block-copolymers. Other examples are silanole-group bearing polyvinylalcoholes available under the trade name Poval® R (Kuraray Europe GmbH; Frankfurt; Germany).

Suitable cationic polymers are polyalkylenepolyamines, cationic derivatives of polyacrylamides, polyethyleneimines and polyquaternary amines.

Polyquaternary amines are, for example, condensation products of hexamethylenediamine, dimethylamine and epichlorohydrin, condensation products of dimethylamine and epichlorohydrin, copolymers of hydroxyethylcellulose and diallyldimethylammonium chloride, copolymers of acrylamide and a methacryloyloxyethyltrimethylammonium chloride, condensation products of hydroxyethylcellulose, epichlorohydrin and trimethylamine, homopolymers of diallyldimethylammonium chloride and addition products of epichlorohydrin to amidoamines. In addition, polyquaternary amines can be obtained by reacting dimethyl sulfate with polymers such as polyethyleneimines, copolymers of vinylpyrrolidone and dimethylaminoethyl methacrylate or copolymers of ethyl methacrylate and diethylaminoethyl methacrylate. The polyquaternary amines are available within a wide molecular weight range.

However, it is also possible to generate the cationic polymers on the particle surface, either through reagents which can form a network with themselves, such as addition products of epichlorohydrin to polyamidoamines, or through the application of cationic polymers which can react with an added crosslinker, such as polyamines or polyimines in combination with polyepoxides, polyfunctional esters, polyfunctional acids or polyfunctional (meth)acrylates.

It is possible to use all polyfunctional amines having primary or secondary amino groups, such as polyethyleneimine, polyallylamine and polylysine. The liquid sprayed by the process according to the invention preferably comprises at least one polyamine, for example polyvinylamine or a partially hydrolyzed polyvinylformamide.

The cationic polymers may be used as a solution in an aqueous or water-miscible solvent, as dispersion in an aqueous or water-miscible dispersant or in substance.

When the water-absorbent polymer particles are coated with a cationic polymer, the use amount of cationic polymer based on the water-absorbent polymer particles is usually not less than 0.001% by weight, typically not less than 0.01% by weight, preferably from 0.1 to 15% by weight, more preferably from 0.5 to 10% by weight, most preferably from 1 to 5% by weight.

Suitable anionic polymers are polyacrylates (in acidic form or partially neutralized as salt), copolymers of acrylic acid and maleic acid available under the trade name Sokalan® (BASF SE; Ludwigshafen; Germany), and polyvinylalcohols with built in ionic charges available under the trade name Poval® K (Kuraray Europe GmbH; Frankfurt; Germany).

Suitable polyvalent metal cations are Mg²⁺, Ca²⁺, Al³⁺, Sc³⁺, Ti⁴⁺, Mn²⁺, Fe^{2+/3+}, Co²⁺, Ni²⁺, Cu^{+/2+}, Zn²⁺, Y³⁺, Zr⁴⁺, Ag⁺, La³⁺, Ce⁴⁺, Hf⁴⁺ and Au^{+/3+}; preferred metal cations are Mg²⁺, Ca²⁺, Ti⁴⁺, Zr⁴⁺ and La³⁺; particularly preferred metal cations are Al³⁺, Ti⁴⁺ and Zr⁴⁺. The metal cations may be used either alone or in a mixture with one another. Suitable metal salts of the metal cations mentioned are all of those which have a sufficient solubility in the solvent to be used. Particularly suitable metal salts have weakly complexing anions, such as chloride, hydroxide, carbonate, acetate, formate, propionate, nitrate, sulfate and methanesulfate. The metal salts are preferably used as a solution or as a stable aqueous colloidal dispersion. The solvents used for the metal salts may be water, alcohols, ethylenecarbonate, propylenecarbonate, dimethylformamide, dimethyl sulfoxide and mixtures thereof. Particularly preference is given to water and water/alcohol mixtures, such as water/methanol, water/isopropanol, water/1,3-propanediole, water/1,2-propandiole/1,4-butanediole or water/propylene glycol.

When the water-absorbent polymer particles are coated with a polyvalent metal cation, the amount of polyvalent metal cation used, based on the water-absorbent polymer particles, is preferably from 0.05 to 5% by weight, more preferably from 0.1 to 1.5% by weight, most preferably from 0.3 to 1% by weight.

Suitable reducing agents are, for example, sodium sulfite, sodium hydrogensulfite (sodium bisulfite), sodium dithionite, sulfinic acids and salts thereof, ascorbic acid, sodium hypophosphite, sodium phosphite, and phosphinic acids and salts thereof. Preference is given, however, to salts of hypophosphorous acid, for example sodium hypophosphite, salts of sulfinic acids, for example the disodium salt of 2-hydroxy-2-sulfinatoacetic acid, and addition products of aldehydes, for example the disodium salt of 2-hydroxy-2 sulfonatoacetic acid. The reducing agent used can be, however, a mixture of the sodium salt of 2-hydroxy-2-sulfinatoacetic acid, the disodium salt of 2-hydroxy-2 sulfonatoacetic acid and sodium bisulfite. Such mixtures are obtainable as Brüggolite® FF6 and Brüggolite® FF7 (Brüggemann Chemicals; Heilbronn; Germany). Also useful is the purified 2-hydroxy-2 sulfonatoacetic acid and its sodium salts, available under the trade name Blancolen® from the same company. The reducing agents are typically used in the form of a solution in a suitable solvent, preferably water. The reducing agent may be used as a pure substance or any mixture of the above reducing agents may be used.

When the water-absorbent polymer particles are coated with a reducing agent, the amount of reducing agent used, based on the water-absorbent polymer particles, is preferably from 0.01 to 5% by weight, more preferably from 0.05 to 2% by weight, most preferably from 0.1 to 1% by weight.

Suitable polyols are polyethylene glycols having a molecular weight of from 400 to 20000 g/mol, polyglycerol, 3- to 100-tuply ethoxylated polyols, such as trimethylolpropane, glycerol, sorbitol, mannitol, inositol, pentaerythritol and neopentyl glycol. Particularly suitable polyols are 7- to 20-tuply ethoxylated glycerol or trimethylolpropane, for example Polyol TP 70® (Perstorp AB, Perstorp, Sweden). The latter have the advantage in particular that they lower the surface tension of an aqueous extract of the water-absorbent polymer particles only insignificantly. The polyols are preferably used as a solution in aqueous or water-miscible solvents.

The polyol can be added before, during, or after surface-crosslinking. Preferably it is added after surface-cross linking. Any mixture of the above listed poyols may be used.

When the water-absorbent polymer particles are coated with a polyol, the use amount of polyol, based on the water-absorbent polymer particles, is preferably from 0.005 to 2% by weight, more preferably from 0.01 to 1% by weight, most preferably from 0.05 to 0.5% by weight.

The coating is preferably performed in mixers with moving mixing tools, such as screw mixers, disk mixers, paddle mixers and drum coater. Suitable mixers are, for example, horizontal Pflugschar® plowshare mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; US) and Ruberg continuous flow mixers (Gebrüder Ruberg GmbH & Co KG, Nieheim, Germany). Moreover, it is also possible to use a fluidized bed for mixing.

According to the described production process fine water-absorbent polymer particles are typically removed also after coating and collected according to the present invention and agglomerated to result in water-absorbent polymer particles G. For agglomeration the fine water-absorbent polymer particles are preferably blended with fine water-absorbent polymer particles collected in at least one different step of the production process.

### Agglomeration

According to the described production process fine water-absorbent polymer particles are typically removed after polymerization and/or post-crosslinking and/or coating.

According to the invention these fine water-absorbent polymer particles are used for producing water-absorbent polymer particles G. Therefore, these rather fine water-absorbent polymer particles are collected. The fine water-absorbent polymer particles which are agglomerated are non-surface crosslinked and/or surface-crosslinked and/or coated or preferably be a mixture of at least two of them.

The water-absorbent polymer particles G are obtainable by agglomerating non-surface post-crosslinked fine water-absorbent polymer particles, surface post-crosslinked fine water-absorbent polymer particles or preferably by agglomerating a blend of non-surface post-crosslinked fine water-absorbent polymer particles and surface post-crosslinked fine water-absorbent polymer particles drying, grinding, sieving and classification of the agglomerated water-absorbent polymer particles. Alternatively by agglomerating a blend of non-surface post-crosslinked fine water-absorbent polymer particles and/or surface post-crosslinked fine water-absorbent polymer and/or coated fine water-absorbent polymer particles, drying, grinding, sieving and classification of the agglomerated water-absorbent polymer particles.

It is preferred, that this mixture or blend comprises at least 50% by weight of non-surface post-crosslinked fine water-absorbent polymer particles and at maximum 50% by weight of surface post-crosslinked fine water-absorbent polymer particles based on the sum of fine polymer particles.

More preferably at least 60% by weight of non-surface post-crosslinked fine water-absorbent polymer particles and at maximum 40% by weight of surface post-crosslinked fine water-absorbent polymer particles. Most preferably at least 66% by weight of non-surface post-crosslinked fine water-absorbent polymer particles and at maximum 34% by weight of surface post-crosslinked fine water-absorbent polymer particles.

It is furthermore preferred that these mixture comprises at least 70% by weight of non-surface post-crosslinked fine water-absorbent polymer particles and at maximum 30% by weight of surface post-crosslinked fine water-absorbent polymer particles, preferably the mixture or blend comprises at least 75% by weight of non-surface post-crosslinked fine water-absorbent polymer particles and at maximum 25% by weight of surface post-crosslinked fine water-absorbent polymer particles.

The performance of the agglomeration is known to those skilled in the art and is not subject to any restrictions.

According to the invention for the agglomeration of the fine water-absorbent polymer particles a solution or suspension comprising,
a) 0.04 to 1.2% by weight water-soluble or water-dispersible polymeric binders or a mixture thereof, based on the water-absorbent polymer particles,
b) 20 to 70% by weight of water based on the water-absorbent polymer particles, and
c) 5 to 20% by weight of a water-miscible organic solvent based on the water-absorbent polymer particles,
   is used.

According to one embodiment of the invention, for agglomeration the solution or suspension is sprayed onto the fine water-absorbent polymer particles. The spraying with the solution or suspension can, for example, be carried out in mixers having moving mixing implements, such as screw mixers, paddle mixers, disk mixers, plowshare mixers and shovel mixers. Useful mixers include for example Lodige® mixers, Bepex® mixers, Nauta® mixers, Processall® mixers and Schugi® mixers. Vertical mixers are preferred. Fluidized bed apparatuses are particularly preferred.

The solution or suspension preferably comprises water and water-miscible organic solvents, such as alcohols, tetrahydrofuran and acetone; water-soluble and/or water-dispersible polymeric binders are used in addition.

Examples of water-soluble polymeric binders may include, but are not limited to: carboxymethyl cellulose, starch, dextran, polyvinylamine, polyethyleneimine, polyvinylalcohol, polyacrylic acid and its salts, polyethylene oxide, polyethyleneglycol and chitosan.

Water-dispersible polymeric binders according to the invention may include, but are not limited to: homo- and copolymers of vinyl esters, in particular vinyl acetate homopolymers and vinyl acetate copolymers with ethylene, acrylates, maleic acid esters, vinyl-amides, other vinylacyl derivatives and/or homo- and co-polymers of acrylic and methacrylic acid esters, such as copolymers of methyl methacrylate, n-butyl acrylate, or 2-ethylhexyl acrylate.

Copolymers based on vinyl esters, acrylic acid esters, and methacrylic acid esters comprise comonomers, for example, styrene, butadiene, a vinylamide, an olefinically unsaturated carboxylic acid or derivatives thereof, a vinylphosphonic acid or derivative thereof, or a (poly)glycol ester of unsaturated acids. Examples of vinylamides include, but are not limited to, N-vinylformamide, N-vinyl-N-methylacetamide, and N-vinylpyrrolidone.

Examples of olefinically unsaturated carboxylic acids are, for example, acrylic acid, methacrylic acid, itaconic acid, and maleic acid. Examples of derivatives of these olefinically unsaturated carboxylic acids are, for example, amides, such as (meth)acrylamide, N-tert-butyl(meth)acrylamide, and N-isopropyl (meth)acryl-amide, and the N-methylolamides or ethers of N-methylolamides, hemiamides, and imides of aliphatic amines, as well as acrylonitrile. Examples of derivatives of vinylphosphonic acid are, for example, the mono- and diesters of C1-C18 alcohols, for example, the methyl, propyl, or stearyl esters.

Glycol esters of unsaturated acids include hydroxyethyl (meth)acrylate or esters of acrylic and methacrylic acid with polyalkylene oxide compounds of the general formula

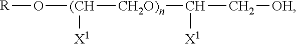

wherein X¹ is hydrogen or methyl, n is 0 to 50, and R is an alkyl, alkaryl, or cycloalkyl C₁-C₂₄ radical, for example, octylphenyl, dodecyl, or nonylphenyl.

Other suitable water-dispersible polymeric binders are polyacetals, i.e., reaction products of polyvinyl alcohols with aldehydes, such as, for example, butyraldehyde; polyurethane polymers prepared from polyhydric alcohols and isocyanates, for example, prepared from polyester and/or polyether diols and, for example, toluene-2,4- or 2,6-diiso-cyanate, methylene-4,4-di(phenyl isocyanate), or hexamethylene diisocyanate; polyureas, i.e., polymers prepared from diamines and diisocyanates or by polycondensation of diamines with carbon dioxide, phosgene, carboxylic acid esters (for example, activated diphenyl carbonates), or urea, or by reaction of diisocyanates with water; a polysiloxane, i.e., linear dimethylpolysiloxane having end groups blocked in different ways; polyamides and copolyamides; polyesters, i.e., polymers prepared by ring-opening polymerization of lactones or by condensation of hydroxycarboxylic acids or diols and dicarboxylic acid derivatives; epoxy resins prepared from polyepoxides by addition reactions with suitable curing agents or by polymerization by way of epoxide groups; polycarbonates prepared by reaction of diglycols or bisphenols with phosgene or carbonic acid diesters in condensation or transesterification reactions; and mixtures thereof.

Preferred water-dispersible polymeric binders are homo- and co-polymers of acrylic acid esters and methacrylic acid esters, and polymers based on polyacetals. Mixtures of two or more polymers also can be used. The mixture ratios are noncritical and are judiciously determined by persons skilled in the art to fit the particular circumstances.

According to the invention a dispersion containing water-dispersible polymeric binders comprising about 5% to about 75%, by weight, of the polymer in water and/or suitable water-miscible organic solvents. The polymer is dispersed in a sufficient amount of water and/or suitable water-miscible organic solvents to allow the polymer to be readily and homogeneously applied to the surfaces of the fine water-absorbent polymer particles. The suitable water-miscible organic solvents for the polymer can be, but is not limited to, an alcohol, or a glycol, such as methanol, ethanol, ethylene glycol, or propylene glycol, and mixtures thereof. Often, the water-dispersible polymeric binders are applied as an emulsion containing the polymer, water, optional organic solvents, emulsifiers, and other ingredients typically used in the preparation of emulsions.

Further suitable organic solvents include, but are not limited to, aliphatic and aromatic hydrocarbons, alcohols, ethers, esters, and ketones, for example, n-hexane, cyclohexane, toluene, xylene, methanol, ethanol, i-propanol, ethylene glycol, 1,2-propanediol, glycerol, diethyl ether, methyltriglycol, a polyethylene glycol having an average molecular weight (Mw) of 200-10,000, ethyl acetate, n-butyl acetate, acetone, 2-butanone, and mixtures thereof.

According to the invention it is preferred that, 0.04 to 0.8% by weight of water-soluble or water-dispersible polymeric binders or a mixture thereof, preferably polyacrylates are used for agglomeration, more preferably from 0.04 to 0.42% by weight, most preferably from 0.16 to 0.42% by weight based on the sum of fine water-absorbent polymer particles.

It is further preferred that 20 to 70% by weight of water are used, more preferably 30 to 60% by weight based on the sum of fine water-absorbent polymer particles.

Furthermore preferably 5 to 20% by weight of a water-miscible organic solvent are present, more preferably from 5 to 15% by weight, most preferably from 5 to 10% by weight based on the sum of fine water-absorbent polymer particles.

The features of the resulting agglomerated water-absorbent polymer particles G are controlled or influenced by the type and amount of binder or the amount of water used in the agglomeration process. An increase of the amount of water e.g. results in a higher amount of resulting particles/agglomerates with a size above 150 μm.

Furthermore, the resulting agglomerated water-absorbent polymer particles G can be e.g. surface-post-crosslinked and/or coated to further adjust their properties.

The average particle diameter of the agglomerated water-absorbent particles G is preferably from 200 to 550 μm, more preferably from 250 to 500 μm, most preferably from 350 to 450 μm.

The resulting water-absorbent polymer particles G having a blood acquisition time of less than 30 s, wherein the blood acquisition time is measured according to the Blood Acquisition Time test method. Preferably the blood acquisition time of the water-absorbent polymer particles G is less than 26 s, more preferably less than 25 s.

According to the invention the resulting water-absorbent polymer particles G having a milk absorption time of less than 15 s, wherein the milk absorption time is measured according to the Milk Absorption Time test method. Preferably the milk absorption time of the water-absorbent polymer particles G is less than 13 s, more preferably less than 12 s.

The water-absorbent polymer particles G have an absorption under load of 0.3 psi (AAP 0.3 psi or AUL 0.3 psi) of at least 10 g/g, preferably of at least 15 g/g, more preferably of at least 18 g/g, most preferably of at least 20 g/g.

The bulk density of the water-absorbent particles G is less than 0.55 g/ml, preferably less than 0.50 g/ml, more preferably less than 0.49 g/ml, most preferably less than 0.47 g/ml. Preferably the bulk density of the water-absorbent particles G is between 0.41 g/ml and 0.49 g/ml.

Water-absorbent polymer particles G prepared by agglomeration according to the invention having a centrifuge retention capacity (CRC) of at least 15 g/g, more preferably of at least 18 g/g, most preferably of at least 20 g/g.

The water-absorbent polymer particles G according to the invention produced by agglomeration of such fine water-absorbent polymer particles having a vortex of less than 15 s and a bulk density of 0.49 g/ml or lower, a CRC of at least 15 g/g, an AUL 0.3 psi of at least 10 g/g, a blood acquisition time of less than 30 s and a milk absorption time of less than 15 s.

### C. Feminine Hygiene Absorbent Articles

The feminine hygiene absorbent article comprises
(A) an upper liquid-pervious layer
(B) a lower liquid-impervious layer
(C) a fluid-absorbent core between (A) and (B) comprising from 50 to 95% by weight a fibrous material and from 5 to 50% by weight water-absorbent polymer particles G;
   or from 60 to 95% by weight a fibrous material and from 5 to 40% by weight water-absorbent polymer particles G;
   preferably from 75 to 95% by weight a fibrous material and from 5 to 25% by weight water-absorbent polymer particles G;
   more preferably from 80 to 90% by weight a fibrous material and from 10 to 20% by weight water-absorbent polymer particles G;
   most preferably from 85 to 90% by weight a fibrous material and from 10 to 15% by weight water-absorbent polymer particles G;
(D) an optional acquisition-distribution layer between (A) and (C), comprising from 80 to 100% by weight a fibrous material and from 0 to 20% by weight water-absorbent polymer particles;
   preferably from 85 to 99.9% by weight a fibrous material and from 0.01 to 15% by weight water-absorbent polymer particles;
   more preferably from 90 to 99.5% by weight a fibrous material and from 0.5 to 10% by weight water-absorbent polymer particles;
   most preferably from 95 to 99% by weight a fibrous material and from 1 to 5% by weight water-absorbent polymer particles;
(E) an optional tissue layer disposed immediately above and/or below (C) or wrapped fully or partially around (C); and
(F) other optional components.

The water-absorbent polymer particles G being obtainable by agglomerating a blend of non-surface post-crosslinked fine water-absorbent particles and surface post-crosslinked fine water-absorbent particles drying, grinding, sieving and classification of the agglomerated water-absorbent polymer particles.

Feminine hygiene absorbent article understood to mean, for example, breast pads, sanitary napkins, pantiliner, or articles for low or moderate adult incontinence, as e.g. incontinence pads and incontinence briefs for adults. Suitable feminine hygiene absorbent articles including fluid-absorbent compositions comprising fibrous materials and optionally water-absorbent polymer particles to form fibrous webs or matrices for the substrates, layers, sheets and/or the fluid-absorbent core.

The acquisition-distribution layer acts as transport and distribution layer of the discharged body fluids and is typically optimized to affect efficient liquid distribution with the underlying fluid-absorbent core. Hence, for quick temporary liquid retention it provides the necessary void space while its area coverage of the underlying fluid-absorbent core must affect the necessary liquid distribution and is adopted to the ability of the fluid-absorbent core to quickly dewater the acquisition-distribution layer.

Suitable feminine hygiene absorbent articles are composed of several layers whose individual elements must show preferably definite functional parameter such as a small insult zone for reasons of less visibility of the absorbed body fluids and dryness for the upper liquid-pervious layer, vapor permeability without wetting through for the lower liquid-impervious layer, a flexible, vapor permeable and thin fluid-absorbent core, showing fast absorption rates and being able to retain highest quantities of body fluids, and an acquisition-distribution layer between the upper layer and the core, acting as transport and distribution layer of the discharged body fluids. These individual elements are combined such that the resultant feminine hygiene absorbent articles meets overall criteria such as flexibility, water vapour breathability, dryness, wearing comfort, softness, less visibility and protection on the user facing side, and concerning liquid retention, rewet and prevention of wet through on the garment side. The specific combination of these layers provides a fluid-absorbent article delivering both high protection levels as well as high comfort to the consumer.

Designs for fluid-absorbent articles and methods to make them are for example described in the following publications and literature cited therein and are expressly incorporated into the present invention: EP 2301499 A1, EP 2314264 A1, EP 2387981 A1, EP 2486901 A1, EP 2524679 A1, EP 2524679 A1, EP 2524680 A1, EP 2565031 A1, U.S. Pat. No. 6,972,011, US 2011/0162989, US 2011/0270204, WO 2010/004894 A1, WO 2010/004895 A1, WO 2010/076857 A1, WO 2010/082373 A1, WO 2010/118409 A1, WO 2010/133529 A2, WO 2010/143635 A1, WO 2011/084981 A1, WO 2011/086841 A1, WO 2011/086842 A1, WO 2011/086843 A1, WO 2011/086844 A1, WO 2011/117997 A1, WO 2011/136087 A1, WO 2012/048879 A1, WO 2012/052173 A1 and WO 2012/052172 A1, WO 2012/009590 A1, WO 2012/047990 A1.

### Liquid-Pervious Layer (A)

Generally the liquid-pervious layer (A) or topsheet is the layer which is in direct contact with the skin. Thus, the liquid-pervious layer is preferably compliant, soft feeling and non-irritating to the consumer's skin. The top sheet suitable for use herein can comprise wovens, non-wovens, and/or three-dimensional webs of a liquid impermeable polymeric film comprising liquid permeable apertures. Generally, the term “liquid-pervious” is understood thus permitting liquids, i.e. body fluids such as urine, menses and/or vaginal fluids to readily penetrate through its thickness. The principle function of the liquid-pervious layer is the acquisition and transport of body fluids from the wearer towards the fluid-absorbent core. The liquid-pervious layer (A) may be made of a hydrophobic material to isolate the wearer's skin from liquids which have passed through the layer. If the liquid-pervious layer (A) is made of a hydrophobic material, at least the upper surface of the liquid-pervious layer (A) is treated to be hydrophilic so that liquids will transfer through the topsheet more rapidly. This diminishes the likelihood that body exudates will flow off the topsheet rather than being drawn through the liquid-pervious layer (A) and being absorbed by the absorbent core. Furthermore it is possible that the liquid-pervious layer can be rendered hydrophilic by treating it with a surfactant. Suitable methods for treating the liquid-pervious layer (A) with a surfactant include spraying the topsheet material with the surfactant and immersing the material into the surfactant.

The liquid-pervious layers for use herein can be a single layer or may have a multiplicity of layers. Typically liquid-pervious layers are formed from any materials known in the art such as nonwoven material, films, such as apertured formed thermoplastic films, apertured plastic films, and hydro formed thermoplastic films; porous foams; reticulated foams; reticulated thermoplastic films; and thermoplastic scrims, or combinations thereof. Suitable liquid-pervious layers (A) consist of customary synthetic or semisynthetic fibers or bicomponent fibers or films of polyester, polyolefins, rayon or natural fibers or any combinations thereof. In the case of nonwoven materials, the fibers should generally be bound by binders such as polyacrylates. Additionally the liquid-pervious layer may contain elastic compositions thus showing elastic characteristics allowing to be stretched in one or two directions.

One suitable material for liquid-pervious layers can be a thermobonded carded web which is available as Code No. P-8 from Fiberweb North America, Inc. (Simpsonville, S.C., U.S.A.). Another suitable topsheet material is available as Code No. S-2355 from Havix Co., Japan. Yet another suitable topsheet material can be a thermobonded carded web which is available as Code No. Profleece Style 040018007 from Amoco Fabrics, Inc. (Gronau, Germany).

Suitable synthetic fibers are made from polyvinyl chloride, polyvinyl fluoride, polytetrafluorethylene, polyvinylidene chloride, polyacrylics, polyvinyl acetate, polyethylvinyl acetate, non-soluble or soluble polyvinyl alcohol, polyolefins such as polyethylene, polypropylene, polyamides, polyesters, polyurethanes, polystyrenes and the like.

Examples for films are liquid permeable, apertured formed thermoplastic films, apertured plastic films, hydroformed thermoplastic films, reticulated thermoplastic films, porous foams, reticulated foams, and thermoplastic scrims. They provide a resilient three-dimensional fibre-like structure. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991 and for example in U.S. Pat. Nos. 4,151,240, 4,319,868, 4,343,314, 4,591,523, 4,609,518, 4,629,643, 4,695,422 or WO 96/00548.

Examples of suitable modified or unmodified natural fibers include cotton, bagasse, kemp, flax, silk, wool, wood pulp, chemically modified wood pulp, jute, rayon, ethyl cellulose, and cellulose acetate.

Suitable wood pulp fibers can be obtained by chemical processes such as the Kraft and sulfite processes, as well as from mechanical processes, such as ground wood, refiner mechanical, thermo-mechanical, chemi-mechanical and chemi-thermo-mechanical pulp processes. Further, recycled wood pulp fibers, bleached, unbleached, elementally chlorine free (ECF) or total chlorine free (TCF) wood pulp fibers can be used.

The fibrous material may comprise only natural fibers or synthetic fibers or any combination thereof. Preferred materials are polyester, rayon and blends thereof, polyethylene, and polypropylene.

The fibrous material as a component of the fluid-absorbent compositions may be hydrophilic, hydrophobic or can be a combination of both hydrophilic and hydrophobic fibers. The definition of hydrophilic is given in the section “definitions” in the chapter above. The selection of the ratio hydrophilic/hydrophobic and accordingly the amount of hydrophilic and hydrophobic fibers within fluid-absorbent composition will depend upon fluid handling properties and the amount of water-absorbent polymer particles of the resulting fluid-absorbent composition. Such, the use of hydrophobic fibers is preferred if the fluid-absorbent composition is adjacent to the wearer of the feminine hygiene absorbent articles, that is to be used to replace partially or completely the upper liquid-pervious layer, preferably formed from hydrophobic nonwoven materials. Hydrophobic fibers can also be member of the lower breathable, but fluid-impervious layer, acting there as a fluid-impervious barrier.

Examples for hydrophilic fibers are cellulosic fibers, modified cellulosic fibers, rayon, polyester fibers such as polyethylen terephthalate, hydrophilic nylon and the like. Hydrophilic fibers can also be obtained from hydrophobic fibers which are hydrophilized by e. g. surfactant-treating or silica-treating. Thus, hydrophilic thermoplastic fibers derived from polyolefins such as polypropylene, polyamides, polystyrenes or the like by surfactant-treating or silica-treating.

To increase the strength and the integrity of the upper-layer, the fibers should generally show bonding sites, which act as crosslinks between the fibers within the layer.

Technologies for consolidating fibers in a web are mechanical bonding, thermal bonding and chemical bonding. In the process of mechanical bonding the fibers are entangled mechanically, e.g., by water jets (spunlace) to give integrity to the web. Thermal bonding is carried out by means of raising the temperature in the presence of low-melting polymers. Examples for thermal bonding processes are spunbonding, through-air bonding and resin bonding.

Preferred means of increasing the integrity are thermal bonding, spunbonding, resin bonding, through-air bonding and/or spunlace.

In the case of thermal bonding, thermoplastic material is added to the fibers. Upon thermal treatment at least a portion of this thermoplastic material is melting and migrates to intersections of the fibers caused by capillary effects. These intersections solidify to bond sites after cooling and increase the integrity of the fibrous matrix. Moreover, in the case of chemically stiffened cellulosic fibers, melting and migration of the thermoplastic material has the effect of increasing the pore size of the resultant fibrous layer while maintaining its density and basis weight. Upon wetting, the structure and integrity of the layer remains stable. In summary, the addition of thermoplastic material leads to improved fluid permeability of discharged body fluids and thus to improved acquisition properties.

Suitable thermoplastic materials including polyolefins such as polyethylene and polypropylene, polyesters, copolyesters, polyvinyl acetate, polyethylvinyl acetate, polyvinyl chloride, polyvinylidene chloride, polyacrylics, polyamides, copolyamides, polystyrenes, polyurethanes and copolymers of any of the mentioned polymers.

Suitable thermoplastic fibers can be made from a single polymer that is a monocomponent fiber. Alternatively, they can be made from more than one polymer, e.g., bi-component or multicomponent fibers. The term “bicomponent fibers” refers to thermoplastic fibers that comprise a core fiber made from a different fiber material than the shell. Typically, both fiber materials have different melting points, wherein generally the sheath melts at lower temperatures. Bi-component fibers can be concentric or eccentric depending whether the sheath has a thickness that is even or uneven through the cross-sectional area of the bicomponent fiber. Advantage is given for eccentric bi-component fibers showing a higher compressive strength at lower fiber thickness. Further bi-component fibers can show the feature “uncrimped” (unbent) or “crimped” (bent), further bi-component fibers can demonstrate differing aspects of surface lubricity.

Examples of bi-component fibers include the following polymer combinations: polyethylene/polypropylene, polyethylvinyl acetate/polypropylene, polyethylene/polyester, polypropylene/polyester, copolyester/polyester and the like.

Suitable thermoplastic materials have a melting point of lower temperatures that will damage the fibers of the layer; but not lower than temperatures, where usually the fluid-absorbent articles are stored. Preferably the melting point is between about 75° C. and 175° C. The typical length of thermoplastic fibers is from about 0.4 to 6 cm, preferably from about 0.5 to 1 cm. The diameter of thermoplastic fibers is defined in terms of either denier (grams per 9000 meters) or dtex (grams per 10000 meters). Typical thermoplastic fibers have a dtex in the range from about 1.2 to 20, preferably from about 1.4 to 10.

A further mean of increasing the integrity of the fluid-absorbent composition is the spunbonding technology. The nature of the production of fibrous layers by means of spunbonding is based on the direct spinning of polymeric granulates into continuous filaments and subsequently manufacturing the fibrous layer.

Spunbond fabrics are produced by depositing extruded, spun fibers onto a moving belt in a uniform random manner followed by thermal bonding the fibers. The fibers are separated during the web laying process by air jets. Fiber bonds are generated by applying heated rolls or hot needles to partially melt the polymer and fuse the fibers together. Since molecular orientation increases the melting point, fibers that are not highly drawn can be used as thermal binding fibers. Polyethylene or random ethylene/propylene copolymers are used as low melting bonding sites.

Besides spunbonding, the technology of resin bonding also belongs to thermal bonding subjects. Using this technology to generate bonding sites, specific adhesives, based on e.g. epoxy, polyurethane and acrylic are added to the fibrous material and the resulting matrix is thermically treated. Thus the web is bonded with resin and/or thermal plastic resins dispersed within the fibrous material.

As a further thermal bonding technology through-air bonding involves the application of hot air to the surface of the fibrous fabric. The hot air is circulated just above the fibrous fabric, but does not push through the fibrous fabric. Bonding sites are generated by the addition of binders. Suitable binders used in through-air thermal bonding include crystalline binder fibers, bi-component binder fibers, and powders. When using crystalline binder fibers or powders, the binder melts entirely and forms molten droplets throughout the nonwoven's crosssection. Bonding occurs at these points upon cooling. In the case of sheath/core binder fibers, the sheath is the binder and the core is the carrier fiber. Products manufactured using through-air ovens tend to be bulky, open, soft, strong, extensible, breathable and absorbent. Through-air bonding followed by immediate cold calendering results in a thickness between a hot roll calendered product and one that has been though-air bonded without compression. Even after cold calendering, this product is softer, more flexible and more extensible than area-bond hot-calendered material.

Spunlacing (“hydroentanglement”) is a further method of increasing the integrity of a web. The formed web of loose fibers (usually air-laid or wet-laid) is first compacted and prewetted to eliminate air pockets. The technology of spunlacing uses multiple rows of fine high-speed jets of water to strike the web on a porous belt or moving perforated or patterned screen so that the fibers knot about one another. The water pressure generally increases from the first to the last injectors. Pressures as high as 150 bar are used to direct the water jets onto the web. This pressure is sufficient for most of the nonwoven fibers, although higher pressures are used in specialized applications.

The spunlace process is a nonwovens manufacturing system that employs jets of water to entangle fibers and thereby provide fabric integrity. Softness, drape, conformability, and relatively high strength are the major characteristics of spunlace nonwoven.

In newest researches benefits are found in some structural features of the resulting liquid-pervious layers. For example, the thickness of the layer is very important and influences together with its x-y dimension the acquisition-distribution behaviour of the layer. If there is further some profiled structure integrated, the acquisition-distribution behaviour can be directed depending on the three-dimensional structure of the layer. Thus 3D-polyethylene in the function of liquid-pervious layer is preferred.

Thus, suitable liquid-pervious layers (A) are nonwoven layers formed from the fibers above by thermal bonding, spunbonding, resin bonding or through-air bonding. Further suitable liquid-pervious layers are 3D-polyethylene layers and spunlace.

Preferably the 3D-polyethylene layers and spunlace show basis weights from 12 to 22 gsm. Typically liquid-pervious layers (A) extend partially or wholly across the fluid-absorbent structure and can extend into and/or form part of all the preferred sideflaps, side wrapping elements, wings and ears.

### Liquid-Impervious Layer (B)

The liquid-impervious layer (B) or backsheet prevents the exudates absorbed and retained by the fluid-absorbent core from wetting articles which are in contact with the feminine hygiene absorbent articles, as for example bedsheets, pants, pyjamas and undergarments. The liquid-impervious layer (B) may thus comprise a woven or a nonwoven material, polymeric films such as thermoplastic film of polyethylene or polypropylene, or composite materials such as film-coated nonwoven material.

A suitable microporous polyethylene film is manufactured by Mitsui Toatsu Chemicals, Inc., Nagoya, Japan and marketed in the trade as PG-P. A suitable liquid impervious thermoplastic film having a thickness of from about 0.012 mm (0.50 mil) to about 0.051 mm (2.0 mils), for example comprising polyethylene or polypropylene and have a basis weight in the range of 5 gsm to 35 gsm.

Suitable liquid-impervious layers include nonwoven, plastics and/or laminates of plastic and nonwoven and are preferable flexible. Herein, “flexible” refers to materials which are compliant and which will readily conform to the general shape and contours of the wearer's body.

Both, the plastics and/or laminates of plastic and nonwoven may appropriately be breathable, that is, the liquid-impervious layer (B) can permit vapors to escape from the fluid-absorbent material. Thus the liquid-impervious layer has to have a definite water vapor transmission rate and at the same time the level of impermeability. To combine these features, suitable liquid-impervious layers including at least two layers, e.g. laminates from fibrous nonwoven having a specified basis weight and pore size, and a continuous three-dimensional film of e.g. polyvinylalcohol as the second layer having a specified thickness and optionally having pore structure. Such laminates acting as a barrier and showing no liquid transport or wet through. Thus, suitable liquid-impervious layers comprising at least a first breathable layer of a porous web which is a fibrous nonwoven, e.g. a composite web of a meltblown nonwoven layer or of a spunbonded nonwoven layer made from synthetic fibers and at least a second layer of a resilient three dimensional web consisting of a liquid-impervious polymeric film, e.g. plastics optionally having pores acting as capillaries, which are preferably not perpendicular to the plane of the film but are disposed at an angle of less than 90° relative to the plane of the film.

Suitable liquid-impervious layers are permeable for vapor. Preferably the liquid-impervious layer is constructed from vapor permeable material showing a water vapor transmission rate (WVTR) of at least about 100 gsm per 24 hours, preferably at least about 250 gsm per 24 hours and most preferred at least about 500 gsm per 24 hours.

Preferably the liquid-impervious layer (B) is made of nonwoven comprising hydrophobic materials, e.g. synthetic fibers or a liquid-impervious polymeric film comprising plastics e.g. polyethylene. The thickness of the liquid-impervious layer is preferably 15 to 30 μm.

Further, the liquid-impervious layer (B) is preferably made of a laminate of nonwoven and plastics comprising a nonwoven having a density of 12 to 15 gsm and a polyethylene layer having a thickness of about 10 to 20 μm.

The backsheet can be typically positioned adjacent the outer-facing surface of the absorbent core and may be joined thereto by any suitable attachment means known in the art. For example, the backsheet may be secured to the absorbent core by a uniform continuous layer of adhesive, a patterned layer of adhesive, or an array of separate lines, spirals, or spots of adhesive.

The typically liquid-impervious layer (B) extends partially or wholly across the fluid-absorbent structure and can extend into and/or form part of all the preferred sideflaps, side wrapping elements, wings and ears.

### Fluid-Absorbent Core (C)

Generally the fluid-absorbent core (C) can be any absorbent member which is generally compressible, conformable, non-irritating to the wearer's skin, and capable of absorbing and retaining body fluids. The fluid-absorbent core (C) may be manufactured in a wide variety of sizes and shapes (e.g., rectangular, hourglass, “T”-shaped, asymmetric, etc.) and from a wide variety of liquid-absorbent materials commonly used in feminine hygiene absorbent articles such as comminuted wood pulp, creped cellulose wadding; meltblown polymers including coform; chemically stiffened, modified or cross-linked cellulosic fibers; tissue including tissue wraps and tissue laminates; absorbent foams; absorbent sponges; water-absorbent polymer particles, absorbent gelling materials; or any equivalent material or combinations of materials.

The configuration and construction of the fluid-absorbent core (C) may vary (e.g., the absorbent core may have varying caliper zones, a hydrophilic gradient, a superabsorbent graclient, or lower average density and lower average basis weight acquisition zones; or may include one or more layers or structures). Further, the size and absorbent capacity of the fluid-absorbent core (C) may also be varied. However, the total absorbent capacity of the fluid-absorbent core (C) should be compatible with the design loading and the intended use of the feminine hygiene absorbent article.

The fluid-absorbent core (C) may include other optional components. One such optional component is the core wrap, i.e., a material, typically but not always a nonwoven material, which either partially or totally surrounds the core. Suitable core wrap materials include, but are not limited to, cellulose, hydrophilic ally modified nonwoven materials, perforated films and combinations thereof.

The fluid-absorbent core (C) is disposed between the upper liquid-pervious layer (A) and the lower liquid-impervious layer (B). Suitable fluid-absorbent cores (C) may be selected from any of the fluid-absorbent core-systems known in the art provided that requirements such as vapor permeability, flexibility and thickness are met. Suitable fluid-absorbent cores refer to any fluid-absorbent composition whose primary function is to acquire, transport, distribute, absorb, store and retain discharged body fluids, especially proteinaceous or serous body fluids.

The top view area of the fluid-absorbent core (C) is preferably at least 50 cm², alternatively at least 100 cm², at least 150 cm² or preferably at least 200 cm² depending on the intended use of the feminine hygiene absorbent article. The top view area is the part of the core that is face-to-face to the upper liquid-pervious layer.

According to the present invention the fluid-absorbent core can include the following components:
1. an optional core cover
2. a fluid storage layer
3. an optional dusting layer

### 1. Optional Core Cover

In order to increase the integrity of the fluid-absorbent core, the core is provided with a cover. This cover may be at the top and/or at the bottom of the fluid-absorbent core with bonding at lateral juncture and/or bonding at the distal juncture by hot-melt, ultrasonic bonding, thermal bonding or combination of bonding techniques know to persons skilled in the art. Further, this cover may include the whole fluid-absorbent core with a unitary sheet of material and thus function as a wrap. Wrapping is possible as a full wrap, a partial wrap or as a C-Wrap.

The material of the core cover may comprise any known type of substrate, including webs, garments, textiles, films, tissues and laminates of two or more substrates or webs. The core cover material may comprise natural fibers, such as cellulose, cotton, flax, linen, hemp, wool, silk, fur, hair and naturally occurring mineral fibers. The core cover material may also comprise synthetic fibers such as rayon and lyocell (derived from cellulose), polysaccharides (starch), polyolefin fibers (polypropylene, polyethylene), polyamides, polyester, butadiene-styrene block copolymers, polyurethane and combinations thereof. Preferably, the core cover comprises synthetic fibers or tissue.

The fibers may be mono- or multicomponent. Multicomponent fibers may comprise a homopolymer, a copolymer or blends thereof.

### 2. Fluid-Storage Layer

Generally, the fluid-absorbent compositions included in the fluid-absorbent core comprise fibrous materials and water-absorbent polymer particles.

Fibers useful in the present invention include natural fibers and synthetic fibers. Examples of suitable modified or unmodified natural fibers are given in the chapter “Liquid-pervious Layer (A)” above.

Examples of suitable synthetic fibers are given in the chapter “Liquid-pervious Layer (A)” above. The fibrous material may comprise only natural fibers or synthetic fibers or any combination thereof.

The fibrous material as a component of the fluid-absorbent compositions may be hydrophilic, hydrophobic or can be a combination of both hydrophilic and hydrophobic fibers.

Generally for the use in a fluid-absorbent core, which is the embedded between the upper layer (A) and the lower layer (B), hydrophilic fibers are preferred. This is especially the case for fluid-absorbent compositions that are desired to quickly acquire, transfer and distribute discharged body fluids to other regions of the fluid-absorbent composition or fluid-absorbent core. The use of hydrophilic fibers is especially preferred for fluid-absorbent compositions comprising water-absorbent polymer particles.

Examples for hydrophilic fibers are given in the chapter “Liquid-pervious Layer (A)” above. Preferably, the fluid-absorbent core is made from viscose acetate, polyester and/or polypropylene.

The fibrous material of the fluid-absorbent core may be uniformly mixed to generate a homogenous or in-homogenous fluid-absorbent core. Alternatively, the fibrous material may be concentrated or laid in separate layers optionally comprising water-absorbent polymer material or alternatively the fibrous material may be concentrated in layers alternating with layers water-absorbent polymer material. Suitable storage layers of the fluid-absorbent core comprising homogenous mixtures of fibrous materials comprising water-absorbent polymer material. Suitable storage layers of the fluid-absorbent core including a layered core-system comprise homogenous mixtures of fibrous materials and comprise water-absorbent polymer material, whereby each of the layers may be built from any fibrous material by means known in the art. The sequence of the layers may be directed such that a desired fluid acquisition, distribution and transfer results, depending on the amount and distribution of the inserted fluid-absorbent material, e.g. water-absorbent polymer particles. Preferably there are discrete zones of highest absorption rate or retention within the storage layer of the fluid-absorbent core, formed of layers or in-homogenous mixtures of the fibrous material, acting as a matrix for the incorporation of water-absorbent polymer particles. The zones may extend over the full area or may form only parts of the fluid-absorbent core.

Fluid-absorbent cores comprise fibrous material and fluid-absorbent material. Suitable is any fluid-absorbent material that is capable of absorbing and retaining body fluids or body exudates such as cellulose wadding, modified and unmodified cellulose, crosslinked cellulose, laminates, composites, fluid-absorbent foams, materials described as in the chapter “Liquid-pervious Layer (A)” above, water-absorbent polymer particles and combinations thereof.

Typically, the fluid-absorbent cores contain a single type of water-absorbent polymer particles or may contain water-absorbent polymer particles derived from different kinds of water-absorbent polymer material. Thus, it is possible to add water-absorbent polymer particles from a single kind of polymer material or a mixture of water-absorbent polymer particles from different kinds of polymer materials, e.g. a mixture of regular water-absorbent polymer particles, derived from gel polymerization with water-absorbent polymer particles G according to the present invention.

Alternatively, it is possible to mix water-absorbent polymer particles showing different feature profiles. Thus, the fluid-absorbent core may contain water-absorbent polymer particles with uniform pH value, or it may contain water-absorbent polymer particles with different pH values, e.g. two- or more component mixtures from water-absorbent polymer particles with a pH in the range from about 4.0 to about 7.0. Preferably, applied mixtures deriving from mixtures of water-absorbent polymer particles got from gel polymerization or inverse suspension polymerization with a pH in the range from about 4.0 to about 7.0 and water-absorbent polymer particles got from drop polymerization.

Suitable fluid-absorbent cores are also manufactured from loose fibrous materials by adding water-absorbent particles and/or water-absorbent polymer fibers or mixtures thereof. The water-absorbent polymer fibers may be formed from a single type of water-absorbent polymer fiber or may contain water-absorbent polymer fibers from different polymeric materials. The addition of water-absorbent polymer fibers may be preferred for being distributed and incorporated easily into the fibrous structure and remaining better in place than water-absorbent polymer particles. Thus, the tendency of gel blocking caused by contacting each other is reduced. Further, water-absorbent polymer fibers are softer and more flexible.

In the process of manufacturing the fluid-absorbent core, water-absorbent polymer particles are brought together with structure forming compounds such as fibrous matrices. Thus, the water-absorbent polymer particles may be added during the process of forming the fluid-absorbent core from loose fibers. The fluid-absorbent core may be formed by mixing water-absorbent polymer particles with fibrous materials of the matrix at the same time or adding one component to the mixture of two or more other components either at the same time or by continuously adding.

Alternatively, the fluid-absorbent core may be formed by fibrous material concentrated in layers alternating with layers of water-absorbent polymer material.

Suitable fluid-absorbent cores including mixtures of water-absorbent polymer particles and fibrous material building matrices for the incorporation of the fluid-absorbent material. Such mixtures can be formed homogenously, that is all components are mixed together to get a homogenous structure. The amount of the fluid-absorbent materials may be uniform throughout the fluid-absorbent core, or may vary, e.g. between the central region and the distal region to give a profiled core concerning the concentration of fluid-absorbent material.

Techniques of application of the water-absorbent polymer materials into the absorbent core are known to persons skilled in the art and may be volumetric, loss-in-weight or gravimetric. Known techniques include the application by vibrating systems, single and multiple auger systems, dosing roll, weigh belt, fluid bed volumetric systems and gravitational sprinkle and/or spray systems. Further techniques of insertion are falling dosage systems consensus and contradictory pneumatic application or vacuum printing method of applying the fluid absorbent polymer materials.

Preferably drum-forming techniques are used where the fluid-absorbent core is formed in cavities of a drum rotating about a horizontal axis and being fed at a point on its periphery with a flow of water-absorbent polymer particles and/or fluid-absorbent fibers and fibrous material. The cylindrical surface of the drum on which the fluid-absorbent core is formed is surmounted by a hood, into which said flow is fed pneumatically from the top, bottom or tangentially. The inside of the hood may also contain the outlet of a feed duct, from which discrete quantities of additional water-absorbent polymer particles are dispensed by intermittently operating valve means under pressure. However, using prior art drum-forming techniques it is not possible to obtain uniform distribution of discrete quantities of water-absorbent polymer particles. Thus, in order to get profiled structures having different concentrations of water-absorbent polymer particles in discrete areas, it is preferred to use the technique written in WO 2010103453 in detail. Using this unit for the production of absorbent cores, by which a defined proportion of water-absorbent polymer particles are dispensed intermittently and controllably by adjustable elements controlling position and speed of application, it is possible to apply discrete quantities of water-absorbent polymer particles to a circumscribed area of precise geometrical shape.

Suitable fluid-absorbent cores including layers may be formed by subsequently generating the different layers in z-direction.

Alternatively a core-structure for feminine hygiene articles can be formed from two or more preformed layers to get a layered fluid-absorbent core. Alternatively, the layers may be combined in a way that a plurality of chambers are formed, in which separately water-absorbent polymer material is incorporated.

Furthermore, it can be preferred that the water-absorbent polymer particles are placed within the core in discrete regions even without chambers, e.g. supported by at least an adhesive.

Suitable preformed layers are processed as e.g. air-laid, wet-laid, laminate or composite structure.

Alternatively, layers of other materials can be added, e.g. layers of opened or closed celled foams or perforated films. Included are also laminates of at least two layers comprising said water-absorbent polymer material.

Alternatively, a core-structure can be formed from two or more layers, formed of e.g. nonwoven and/or thermoplastic materials containing water-absorbent polymer particles discretely contained in closed pockets. Such structures are preferably used for forming ultrathin absorbent products. The pockets are free of cellulose pulp. The bonds to define pockets are formed e.g. by intersection of ultrasonic contact areas between two thermoplastic containment layers. Further methods of immobilization of particulate fluid-absorbent material as well as the joining of layers in a layered structure are explained later on in more detail.

Alternatively, a core-structure for ultrathin feminine hygiene articles can be formed from absorbent paper, e.g. a thin and flexible single layer of any suitable absorbent material known in the art including, but not limited to, short-fiber air-laid nonwoven materials; nonwoven of materials such as polyethylene, polypropylene, nylon, polyester, and the like; cellulosic fiBrous materials such as paper tissue or towels known in the art, wax-coated papers, corrugated paper materials, and the like; or fluff pulp. The layer is macroscopically two-dimensional and planar and of very low thickness compared to the other dimensions. Said single layer may also incorporate superabsorbent material throughout the layer. Said single layer may further incorporate bi-component binding fibers. It may be also preferred to combine at least two of such layers in a core structure.

Alternatively, said absorbent paper may by formed from more layers, e.g. a layered absorbent sheet comprising a first layer on the wearer side, a second layer on the non-absorbing side and water-absorbent polymer particles in between or coated on one or both sides of the sheet layers.

The absorbent paper layer has a total basis weight ranging from about 100 gsm to about 1000 gsm, preferably from about 200 gsm to about 750 gsm, and more preferably from about 300 gsm to about 600 gsm.

Further a composite structure can be formed from a carrier layer (e.g. a polymer film), onto which the water-absorbent polymer material is affixed. The fixation can be done at one side or at both sides. The carrier layer may be pervious or impervious for body-fluids.

Thus, suitable fluid-absorbent cores according to the invention comprising from 50% to 95% by weight a fibrous material and from 5 to 50% by weight water-absorbent polymer particles G; preferably from 75 to 95% by weight a fibrous material and from 5 to 25% by weight water-absorbent polymer particles G; more preferably from 80 to 90% by weight a fibrous material and from 10 to 20% by weight water-absorbent polymer particles G and most preferably from 85 to 90% by weight a fibrous material and from 10 to 15% by weight water-absorbent polymer particles G.

It is particularly preferred according to the present invention that the fluid-absorbent core of the inventive feminine hygiene absorbent articles comprises at least 5% by weight of water-absorbent polymer particles G, preferably at least 10% by weight of water-absorbent polymer particles G, more preferably at least 15% by weight of water-absorbent polymer particles G, most preferably 20% by weight of water-absorbent polymer particles G.

It is particularly preferred that the fluid-absorbent core comprises at maximum 95% by weight fibrous material, preferred at maximum 90%, more preferred at maximum 80% by weight fibrous material.

According to the invention it is preferred that the fluid-absorbent core comprises not more than 10% by weight of an adhesive.

The quantity of water-absorbent polymer particles G within the fluid-absorbent core is from 0.1 to 10 g in light-incontinence products, and from 0.1 g to 2 g, preferably from 0.35 to 1 g, e.g. in sanitary napkins or breast pads.

The fluid-absorbent core may comprise additional additives typically present in fluid-absorbent articles known in the art. Exemplary additives are fibers for reinforcing and stabilizing the fluid-absorbent core. Preferably polyethylene is used for reinforcing the fluid-absorbent core.

Further suitable stabilizers for reinforcing the fluid-absorbent core are materials acting as binder.

In varying the kind of binder material or the amount of binder used in different regions of the fluid-absorbent core it is possible to get a profiled stabilization. For example, different binder materials exhibiting different melting temperatures may be used in regions of the fluid-absorbent core, e.g. the lower melting one in the central region of the core, and the higher melting in the distal regions. Suitable binder materials may be adhesive or non-adhesive fibers, continuously or discontinuously extruded fibers, bi-component staple fibers, non-elastomeric fibers and sprayed liquid binder or any combination of these binder materials.

Further, thermoplastic compositions usually are added to increase the integrity of the core layer. Thermoplastic compositions may comprise a single type of thermoplastic polymers or a blend of thermoplastic polymers. Alternatively, the thermoplastic composition may comprise hot melt adhesives comprising at least one thermoplastic polymer together with thermoplastic diluents such as tackifiers, plasticizers or other additives, e.g. antioxidants. The thermoplastic composition may further comprise pressure sensitive hot melt adhesives comprising e.g. crystalline polypropylene and an amorphous polyalphaolefin or styrene block copolymer and mixture of waxes.

Suitable thermoplastic polymers are styrenic block copolymers including A-B-A triblock segments, A-B diblock segments and (A-B), radial block copolymer segments. The letter A designs non-elastomeric polymer segments, e.g. polystyrene, and B stands for unsaturated conjugated diene or their (partly) hydrogenated form. Preferably B comprises isoprene, butadiene, ethylene/butylene (hydrogenated butadiene), ethylene/propylene (hydrogenated isoprene) and mixtures thereof.

Other suitable thermoplastic polymers are amorphous polyolefins, amorphous polyalphaolefins and metallocene polyolefins.

Concerning odor control, perfumes and/or odor control additives are optionally added. Suitable odor control additives are all substances of reducing odor developed in carrying fluid-absorbent articles over time known in the art. Thus, suitable odor control additives are inorganic materials, such as zeolites, activated carbon, bentonite, silica, aerosile, kieselguhr, clay; chelants such as ethylenediamine tetraacetic acid (EDTA), cyclodextrins, aminopoly-carbonic acids, ethylenediamine tetramethylene phosphonic acid, aminophosphate, polyfunctional aromates, N,N-disuccinic acid.

Suitable odor control additives are further antimicrobial agents such as quaternary ammonium, phenolic, amide and nitro compounds and mixtures thereof; bactericides such as silver salts, zinc salts, cetylpyridinium chloride and/or triclosan as well as surfactants having an HLB value of less than 12.

Suitable odor control additives are further compounds with anhydride groups such as maleic-, itaconic-, polymaleic- or polyitaconic anhydride, copolymers of maleic acid with C₂-C₈ olefins or styrene, polymaleic anhydride or copolymers of maleic anhydride with isobutene, di-isobutene or styrene, compounds with acid groups such as ascorbic, benzoic, citric, salicylic or sorbic acid and fluid-soluble polymers of monomers with acid groups, homo- or copolymers of C₃-C₅ mono-unsaturated carboxylic acids.

Suitable odor control additives are further perfumes such as allyl caproate, allyl cyclohexaneacetate, allyl cyclohexanepropionate, allyl heptanoate, amyl acetate, amyl propionate, anethol, anixic aldehyde, anisole, benzaldehyde, benzyl acetete, benzyl acetone, benzyl alcohole, benzyl butyrate, benzyl formate, camphene, camphor gum, laevo-carveol, cinnamyl formate, cis-jasmone, citral, citronellol and its derivatives, cuminic alcohol and its derivatives, cyclal C, dimethyl benzyl carbinol and its derivatives, dimethyl octanol and its derivatives, eucalyptol, geranyl derivatives, lavandulyl acetete, ligustral, d-limonene, linalool, linalyl derivatives, menthone and its derivatives, myrcene and its derivatives, neral, nerol, pcresol, p-cymene, orange terpenes, alpha-ponene, 4-terpineol, thymol etc.

Masking agents are also used as odor control additives. Masking agents are in solid wall material encapsulated perfumes. Preferably, the wall material comprises a fluid-soluble cellular matrix which is used for time-delay release of the perfume ingredient.

Further suitable odor control additives are transition metals such as Cu, Ag, Zn; enzymes such as urease-inhibitors, starch, pH buffering material, chitin, green tea plant extracts, ion exchange resin, carbonate, bicarbonate, phosphate, sulfate or mixtures thereof.

Preferred odor control additives are green tea plant extracts, silica, zeolite, carbon, starch, chelating agent, pH buffering material, chitin, kieselguhr, clay, ion exchange resin, carbonate, bicarbonate, phosphate, sulfate, masking agent or mixtures thereof. Suitable concentrations of odor control additives are from about 0.5 to about 300 gsm.

Newest developments propose the addition of wetness indication additives. Besides electrical monitoring the wetness in the feminine hygiene absorbent articles, wetness indication additives comprising a hot melt adhesive with a wetness indicator are known. The wetness indication additive changes the colour from yellow to a relatively dark and deep blue. This colour change is readily perceivable through the liquid-impervious outer material of the fluid-absorbent article. Existing wetness indication is also achieved via application of water soluble ink patterned on the backsheet which disappears when wet.

Suitable wetness indication additives comprising a mixture of sorbitan monooleate and polyethoxylated hydrogenated castor oil. Preferably, the amount of the wetness indication additive is in the range of about 0.0001 to 2% by weight related to the weight of the fluid-absorbent core.

The basis weight of the fluid-absorbent core is in the range of 100 to 1000 gsm, preferably 300 to 600 gsm. The density of the fluid-absorbent core is in the range of 0.1 to 0.25 g/cm³. The thickness of the fluid-absorbent core is in the case sanitary napkins in the range of 1 to 5 mm, preferably 1.5 to 3 mm, in the case of incontinence products in the range of 1 to 15 mm.

### 3. Optional Dusting Layer

An optional component for inclusion into the absorbent core is a dusting layer adjacent to. The dusting layer is a fibrous layer and may be placed on the top and/or the bottom of the absorbent core. Typically, the dusting layer is underlying the storage layer. This underlying layer is referred to as a dusting layer, since it serves as carrier for deposited water-absorbent polymer particles during the manufacturing process of the fluid-absorbent core. If the water-absorbent polymer material is in the form of macrostructures, films or flakes, the insertion of a dusting layer is not necessary. In the case of water-absorbent polymer particles derived from dropletization polymerization, the particles have a smooth surface with no edges. Also in this case, the addition of a dusting layer to the fluid-absorbent core is not necessary. On the other side, as a great advantage the dusting layer provides some additional fluid-handling properties such as wicking performance and may offer reduced inciBence of pin-holing and or pock marking of the liquid impervious layer (B).

Preferably, the dusting layer is a fibrous layer comprising fluff (cellulose fibers). Acquisition-distribution Layer (D)

An optional acquisition-distribution layer (D) is located between the upper layer (A) and the fluid-absorbent core (C) and is preferably constructed to efficiently acquire discharged body fluids and to transfer and distribute them to other regions of the fluid-absorbent composition or to other layers, where the body fluids are immobilized and stored. Thus, the upper layer transfers the discharged liquid to the acquisition-distribution layer (D) for distributing it to the flu-id-absorbent core.

The acquisition-distribution layer (D) comprises fibrous material and optionally water-absorbent polymer particles.

The fibrous material may be hydrophilic, hydrophobic or can be a combination of both hydrophilic and hydrophobic fibers. It may be derived from natural fibers, synthetic fibers or a combination of both.

Suitable acquisition-distribution layers are formed from cellulosic fibers and/or modified cellu-losic fibers and/or synthetics or combinations thereof. Thus, suitable acquisition-distribution layers may contain cellulosic fibers, in particular wood pulp fluff. Examples of fur-ther suitable hydrophilic, hydrophobic fibers, as well as modified or unmodified natural fibers are given in the chapter “Liquid-pervious sheet or liquid pervious layer (A)” above.

Especially for providing both fluid acquisition and distribution properties, the use of modified cellulosic fibers are preferred. Examples for modified cellulosic fibers are chemically treated cellulosicfibers, especially chemically stiffened cellulosic fibers. The term “chemically stiffened cellulosic fibers” means cellulosic fibers that have been stiffened by chemical means to increase the stiffness of the fibers. Such means include the addition of chemical stiffening agent in the form of surface coatings, surface cross-linking and impregnates. Suitable polymeric stiffening agents can include: cationic modified starches having nitrogen-containing groups, latexes, wet strength resins such as polyamide-epichlorohydrin resin, polyacrylamide, urea formaldehyde and melamine formaldehyde resins and polyethylenimine resins

Stiffening may also include altering the chemical structure, e.g. by crosslinking polymer chains. Thus crosslinking agents can be applied to the fibers that are caused to chemically form intrafiber crosslink bonds. Further cellulosic fibers may be stiffened by crosslink bonds in individualized form. Suitable chemical stiffening agents are typically monomeric crosslinking agents including C₂-C₈ dialdehyde, C₂-C₈ monoaldehyde having an acid functionality, and especially C₂-C₉ polycarboxylic acids.

Stiffening may also include altering the chemical structure, e.g. by crosslinking polymer chains. Thus crosslinking agents can be applied to the fibers that are caused to chemically form intrafiber crosslink bonds. Further cellulosic fibers may be stiffened by crosslink bonds in individualized form. Suitable chemical stiffening agents are typically monomeric crosslinking agents including C₂-C₈ dialdehyde, C₂-C₈ monoaldehyde having an acid functionality, and especially C₂-C₉ polycarboxylic acids.

Preferably the modified cellulosic fibers are chemically treated cellulosic fibers. Especially preferred are curly fibers which can be obtained by treating cellulosic fibers with citric acid. Preferably the basis weight of cellulosic fibers and modified cellulosic fibers is from 50 to 200 gsm.

Suitable acquisition-distribution layers further include synthetic fibers. Known examples of synthetic fibers are found in the Chapter “Liquid-pervious sheet or liquid pervious layer (A)” above. Another possibility available is 3D-polyethylene film with dual function as a liquid-pervious layer (A) and acquisition-distribution layer.

Further, as in the case of cellulosic fibers, hydrophilic synthetic fibers are preferred. Hydrophilic synthetic fibers may be obtained by chemical modification of hydrophobic fibers. Prefer-ably, hydrophilization is carried out by surfactant treatment of hydrophobic fibers. Thus the sur-face of the hydrophobic fiber can be rendered hydrophilic by treatment with a nonionic or ionic surfactant, e.g., by spraying the fiber with a surfactant or by dipping the fiber into a surfactant. Further preferred are permanent hydrophilic synthetic fibers.

The fibrous material of the acquisition-distribution layer may be fixed to increase the strength and the integrity of the layer. Technologies for consolidating fibers in a web are mechanical bonding, thermal bonding and chemical bonding. Detailed description of the different methods of increasing the integrity of the web is given in the Chapter “Liquid-pervious sheet or liquid pervious layer (A)” above.

Preferred acquisition-distribution layers comprise fibrous material and water-absorbent polymer particles distributed within. The water-absorbent polymer particles may be added during the process of forming the layer from loose fibers, or, alternatively, it is possible to add monomer solution after the formation of the layer and polymerize the coating solution by means of UV-induced polymerisation technologies. Thus, “in situ”-polymerisation is a further method for the application of water-absorbent polymers.

Thus, suitable acquisition-distribution layers comprising from 80 to 100% by weight a fibrous material and from 0 to 20% by weight water-absorbent polymer particles; preferably from 85 to 99.9% by weight a fibrous material and from 0.1 to 15% by weight water-absorbent polymer particles; more preferably from 90 to 99.5% by weight a fibrous material and from 0.5 to 10% by weight water-absorbent polymer particles; and most preferably from 95 to 99% by weight a fibrous material and from 1 to 5% by weight water-absorbent polymer particles

Alternatively a liquid-impervious layer (D) comprising a synthetic resin film between (A) and (C) acting as an distribution layer and quickly transporting the supplied urine along the surface to the upper lateral portion of the fluid-absorbent core (C). Preferably, the upper liquid-impervious layer (D) is smaller than the underlaying fluid-absorbent core (C). There is no limit in particular to the material of the liquid-impervious layer (D). Such a film made of a resin such as polyethylene, polypropylene, polyethylene therephthalate, polyurethane, or crosslinked polyvinyl alcohol and an air-permeable, but liquid-impervious, so-called: “breathable” film made of above described resin, may be used.

Preferably, the upper liquid-impervious layer (D) comprises a porous polyethylene film for both quick acquisition and distribution of fluid.

Alternatively, a bundle of synthetic fibers acting as acquisition-distribution layer loosely distributed on top of the fluid-absorbent core may be used. Suitable synthetic fibers are of copolyester, polyamide, copolyamide, polylactic acid, polypropylene or polyethylene, viscose or blends thereof. Further bicomponent fibers can be used. The synthetic fiber component may be composed of either a single fiber type with a circular cross-section or a blend of two fibre types with different cross-sectional shapes. Synthetic fibers arranged in that way ensuring a very fast liquid transport and canalisation. Preferably bundles of polyethylene fibers are used.

### Other Optional Components (F)

### 1. Elastics

### 1. Closing or Attachment System

The attachment system comprising sideflaps, side wrapping elements, wings and ears. The side of the attachment system facing the garment, e.g. the underwear may be coated with adhesives, e.g. pressure sensitive adhesive. To protect this coating this attachment system is covered by a layer, e.g. silicon release paper. This paper is removed before use and the article can be fixed at garment e.g. underwear through the adhesive.

The closing or attachment system is either re-sealable or permanent, including any material suitable for such a use, e.g. plastics, elastics, films, foams, nonwoven substrates, woven substrates, paper, tissue, laminates, fiber reinforced plastics, adhesive preferably pressure sensitive adhesives and the like, or combinations thereof. Suitable attachments may be formed of thermoplastic polymers such as polyethylene, polyurethane, polystyrene, polycarbonate, polyester, ethylene vinyl acetate, ethylene vinyl alcohol, ethylene vinyl acetate acrylate or ethylene acrylic acid copolymers.

Suitable closing systems may comprise elastomerics for the production of elastic areas within the fastening devices of the fluid-absorbent article. Elastomerics provide a conformable fit of the fluid-absorbent article to the wearer while maintaining adequate performance against leakage.

Suitable elastomerics are elastomeric polymers or elastic adhesive materials showing vapor permeability and liquid barrier properties. Preferred elastomerics are retractable after elongation to a length equivalent to its original length.

Preferred closing systems are so-called “elastic ears” attached with one side of the ear to the longitudinal side edges located at the rear dorsal longitudinal edge of the chassis of the fluid-absorbent article. Commercially available fluid-absorbent articles include stretchable ears or side panels which are made from a stretchable laminate e.g. nonwoven webs made of mono- or bi-component fibers. Especially preferred closing systems are stretchable laminates comprising a core of several layers each of different fibrous materials, e.g. meltblown fibers, spunbond fibers, containing multicomponent fibers having a core comprising a first polymer having a first melt temperature and a sheath comprising a second polymer having a second melt temperature; and a web of an elastomeric material as top and bottom surfaces to form said laminate.

### 2. Lotions, Flavor or Base Perfume

The feminine absorbent article may comprise additives such as lotion e.g. the topsheet may be lotioned. Lotions of different types are known to provide various skin benefits, such as prevention or treatment of skin rash. These lotions are applied to the top sheet of absorbent articles, for example, and can be transferred to the skin of the wearer during use.

The lotion composition may comprise a plastic or fluid emollient such as mineral oil or petrolatum, an immobilizing agent such as a fatty alcohol or paraffin wax to immobilize the emollient on the surface of the topsheet, and optionally a hydrophilic surfactant to improve wettability of the coated topsheet. Because the emollient is substantially immobilized on the surface of the topsheet, less lotion is required to impart the desired therapeutic or protective lotion coating benefits. The lotion composition may include a rheology structurant selected from the group consisting of microcrystalline wax, alkyl dimethicone, ethylene glycol dibehenate, ethylene glycol distearate, glycerol tribehenate, glycerol tristearate, and ethylene bisoleamide.

The compositions may contain one or more derivatives of essential oil compounds for use in water absorbent articles. These derivatives include acetals of parent essential oil aldehydes and ketones; esters or ethers of parent essential oil alcohols and phenolics; and esters of parent essential oil acids. Examples of parent essential oil aldehydes and ketones include citral, cinnamic aldehyde-anisaldehyde, vanillin, ethyl vanillin, heliotropin, carvone, and menthone. Examples of parent essential oil alcohols and phenolics include thymol, eugenol, isoeugenol, dihydroeugenol, carvacrol, carveol, geraniol, nerol, vanillyl alcohol, heliotropyl alcohol, anisyl alcohol, cinnamyl alcohol and beta-ionol. Examples of parent essential oil acids include, -anisic acid, cinnamic acid, vanillic acid and geranic acid. The present compositions comprising essential oil derivatives are useful as base flavor or base perfume for incorporation into personal care products and to provide other benefits including antimicrobial efficacy. Optionally the compositions will contain additional antimicrobially- or anti-inflammatory-effective components including those also derived from plant essential oils or synthetic versions thereof.

Furthermore, Dihydrobisabolene, Dihydrobisabolol and alpha-Bisabolol may also be used in personal care compositions.

### D. Feminine Hygiene Absorbent Articles Construction

The present invention further relates to the joining of the components and layers, films, sheets, tissues or substrates mentioned above to provide the fluid-absorbent article. At least two, preferably all layers, films, sheets, tissues or substrates are joined.

Suitable feminine hygiene absorbent articles include a single- or multiple fluid-absorbent core-system. Preferably fluid-absorbent articles include a single- or double fluid-absorbent core-system.

Suitable fluid-storage layers of the fluid-absorbent core comprising homogenous or in-homogenous mixtures of fibrous materials comprising water-absorbent polymer particles homogenously or in-homogenously dispersed in it. Suitable fluid-storage layers of the fluid-absorbent core including a layered fluid-absorbent core-system comprising homogenous mixtures of fibrous materials and optionally comprising water-absorbent polymer particles, whereby each of the layers may be prepared from any fibrous material by means known in the art.

In order to immobilize the water-absorbent polymer particles, the adjacent layers are fixed by the means of thermoplastic materials, thereby building connections throughout the whole surface or alternatively in discrete areas of junction. For the latter case, cavities or pockets are built carrying the water-absorbent particles. The areas of junction may have a regular or irregular pattern, e.g. aligned with the longitudinal axis of the fluid-absorbent core or in a pattern of polygons, e.g. pentagons or hexagons. The areas of junction itself may be of rectangular, circular or squared shape with diameters between about 0.5 mm and 2 mm. Fluid-absorbent articles comprising areas of junction show a better wet strength.

The construction of the products chassis and the components contained therein is made and controlled by the discrete application of attachment means e.g. hotmelt adhesives as known to people skilled in the art. Examples would be e.g. Dispomelt 505B, Dispomelt Cool 1101, as well as other specific function adhesives manufactured by Bostik, Henkel or Fuller. Further examples suitable attachment means including an open pattern network of filaments of adhesive are disclosed in U.S. Pat. No. 4,573,986 entitled “Disposable Waste-Containment Garment”, which issued to Minetola et al. on Mar. 4, 1986. Another suitable attachment means including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment means may include heat bonds, pressure bonds, ultrasonic bonds, dynamic mechanical bonds, or any other suitable attachment means or combinations of these attachment means as are known in the art.

In order to ensure wicking of applied body fluids, preferred feminine hygiene absorbent articles show channels for better transport. Channels are formed by compressional forces of e.g. the top sheet against the fluid-absorbent core. Compressive forces may be applied e.g. by heat-treatment between two heated calendar rollers. As an effect of compression both on top sheet and fluid-absorbent core deform such that a channel is created. Body fluids are flowing along this channel to places where they are absorbed and leakage is prevented. Otherwise, compression leads to higher density; this is the second effect of the channel to canalize insulted fluids. Additionally, compressive forces on absorbent article construction improve the structural integrity of the fluid-absorbent article.

A feminine hygiene absorbent article according to the invention, comprising
(A) an upper liquid-pervious layer,
(B) a lower liquid-impervious layer,
(C) a fluid-absorbent core between the layer (A) and the layer (B), comprising 5% to 50% by weight of water-absorbent polymer particles G and not more than 95% by weight of fibrous material, based on the sum of water-absorbent polymer particles G and fibrous material;
(D) an optional acquisition-distribution layer between (A) and (C),
(E) an optional tissue layer disposed immediately above and/or below (C); and
(F) other optional components,
   wherein the water-absorbent polymer particles G being obtainable by agglomerating a blend of non-surface post-crosslinked fine water-absorbent polymer particles and surface post-crosslinked fine water-absorbent polymer particles drying, grinding, sieving and classification of the agglomerated fine water-absorbent polymer particles.

Thus, preferred feminine hygiene absorbent articles are subsequently described in detail. One preferred embodiment of the present invention is described hereinafter.

Thus, a preferred feminine hygiene absorbent article comprising
(A) an upper liquid-pervious layer comprising a spunbond or embossed layer (coverstock)
(B) a lower liquid-impervious layer comprising a composite of polyethylene film and pressure sensitive adhesive
(C) a single fluid-absorbent core (C) between (A) and (B) comprising between 5 to 50% by weight water-absorbent polymer particles based on the total absorbent core weight. According to the invention the fluid-absorbent core comprising water-absorbent polymer particles G.
(D) an acquisition-distribution layer (D) between (A) and (C) having a basis weight of 20 to 80 gsm; the acquisition-distribution layer (D) is rectangular shaped and has the same or smaller size than the fluid-absorbent core.

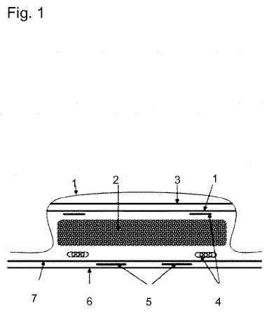

Such an embodiment is schematically shown in FIG. 1.

The reference numerals have the following meanings:
1 liquid-pervious layer (A)
1a Coverstock
2 Fluid-absorbent core (C)
3 Acquisition-distribution layer (ADL) (D)
4 Attachment means
5 Pressure sensitive adhesive
6 Outer poly packaging, e.g. Silicon release paper
7 liquid-impervious layer or backsheet (B)

The construction of the products chassis and the components contained therein is made and controlled by the discrete application of hotmelt adhesives as attachment means (4) as known to people skilled in the art. Examples would be e.g. Dispomelt 505B, Dispomelt Cool 1101, as well as other specific function adhesives manufactured by for example Bostik, Henkel or Fuller.

In order to ensure wicking of applied body fluids, preferred fluid-absorbent article show channels for better transport. Channels are formed by compressional forces of e.g. the top sheet against the fluid-absorbent core. Compressive forces may be applied e.g. by heat treatment between two heated calendar rollers. As an effect of compression both on top sheet and fluid-absorbent core deform such that a channel is created. Body fluids are flowing along this channel to places where they are absorbed and leakage is prevented. Otherwise, compression leads to higher density; this is the second effect of the channel to canalize insulted fluids. Additionally, compressive forces on diaper construction improve the structural integrity of the fluid-absorbent article.

### Methods:

The measurements should, unless stated otherwise, be carried out at an ambient temperature of 23±2° C. and a relative atmospheric humidity of 50±10%. The water-absorbent polymers are mixed thoroughly before the measurement.

The “WSP” standard test methods are described in: “Standard Test Methods for the Nonwovens Industry”, jointly issued by the “Worldwide Strategic Partners” EDANA (European Disposables and Nonwovens Association, Avenue Eugene Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). This publication is available both from EDANA and INDA.

Absorbency Under Load (0.3 psi)

The absorbency under load (AUL or AAP) of the water-absorbent polymer particles is determined analogously to the EDANA recommended test method No. WSP 242.3 (11) “Gravimetric Determination of Absorption Under Pressure”.

### Blood Absorption Time

The blood absorption speed was determined by the time of 1 g super absorbent polymer absorbing 5 ml artificial blood (as described below). Weight 1.000±0.005 g super absorbent polymer into a 100 mL beaker, then 5.0 ml of artificial blood (23±1° C.) is poured into the beaker as quickly as possible, with starting the stop watch synchronously. The stopwatch was stopped when there was no fluid in the beaker, namely, all the liquid was absorbed by the super absorbent polymer. The displayed time (in seconds) was recorded as the blood absorption time

### Preparation of Artificial Blood

The preparation of artificial blood is determined by Standardization Administration of the People's Republic of China recommended preparation method GB/T 22905-2008. By subsequent addition of 10.0 g of NaCl (Sinopharm Chemical Reagent Co., Ltd, China), 40.0 g of Na₂CO₃ (Sinopharm Chemical Reagent Co., Ltd, China), 1.0 g of sodium benzoate (Sinopharm Chemical Reagent Co., Ltd, China) and 5.0 g of sodium carboxymethyl cellulose 800-1200 mPa·s (Sinopharm Chemical Reagent Co., Ltd, China), 140.0 ml of glycerol (Sinopharm Chemical Reagent Co., Ltd, China), and 860.0 g of deionized water into a 2000 ml beaker, a mixture solution is prepared after stirring for 1 hour. Thereafter 10.0 ml of standard media (National Paper Standardization Center, China) and 0.1 g of brilliant blue (Shanghai Dyestuffs Research Institute Co., Ltd., China) are added and the mixture is stirred for more than 24 hours before use. The final artificial blood physical properties should meet the requirements from Table 1.

TABLE 1
Physical property requirements
of artificial blood at 23 ± 1° C.

| Property | Required data | | Standard test number |
|---|---|---|---|
| Density | (1.05 ± 0.05) | g/ml | ISO 758 |
| Viscosity | (7.3 ± 1.1) | mPa · s | ISO 6388 |
| Surface tension | (40 ± 4) | mN/m | EN 14370 |

| | | |
|---|---|---|
| pH value | 11.0 ± 0.1 | ISO 6353-1 |

### Bulk Density

The bulk density of the water-absorbent polymer particles is determined by the EDANA recommended test method No. WSP 250.3 (11) “Gravimetric Determination of Density”

### Centrifuge Retention Capacity (CRC)

The centrifuge retention capacity of the water-absorbent polymer particles is determined by the EDANA recommended test method No. WSP 241.3 (11) “Fluid Retention Capacity in Saline, After Centrifugation”, wherein for higher values of the centrifuge retention capacity larger tea bags have to be used.

### Density of a Fluid-Absorbent Core

This test determines the density of the fluid-absorbent core in the point of interest.

The fluid-absorbent article is clamped nonwoven side up onto the inspection table. The insult point is marked on the article accordingly.

Next, three readings of thickness of the total core are taken using a Portable Thickness Gauge Model J100 (SDL Atlas, Inc.; Stockport; UK) and the average is recorded (T). The weight of the core is recorded (WT).

In case the core is not rectangular shaped a section (e.g. 6 cmX core width) is marked on the fluid-absorbent core with the insult point in the centre of the section. Three readings of thickness of the section are taken using a Portable Thickness Gauge Model J100 (SDL Atlas, Inc.; Stockport; UK) and the average is recorded (T). The section of the fluid-absorbent core is cut out of and the weight of the cut out section is recorded (WT).

The density of the fluid-absorbent core is calculated as follows:

Density[g/cm³]=WT/(area of the section/core×T)

### Milk Absorption Time

The milk absorption time was determined by the time 1 g super absorbent polymer absorbs 10 ml of formulated milk solution. The formulated milk solution was prepared by weighing 10.000±0.005 g of formulated milk powder (Aptamil Pre, Germany) and dissolving it into 60.0 ml of deionized water. Weight 1.000±0.005 g super absorbent polymer into a 50 mL beaker, then 10.0 ml of formulated milk solution (23±1° C.) was poured into the beaker as quickly as possible, with starting the stop watch synchronously. The stopwatch was stopped when there was no fluid in the beaker, namely, all the liquid was absorbed by the super absorbent polymer. The displayed time (in seconds) was recorded as the milk absorption time

### Particle Size Distribution (PSD)

The particle size distribution of the water-absorbent polymer particles is determined by the EDANA recommended test method No. WSP 220.3 (11) “Particle Size Distribution”.

The average particle diameter or also referred to as mean particle size (d₅₀) here is the value of the mesh size which gives rise to a cumulative 50% by weight.

The degree of polydispersity a of the particle size particle is calculated by

α=(d_{84.13}−d_{15.87})/(2×d₅₀)

wherein d_{15.87} and d_{84.13} is the value of the mesh size which gives rise to a cumulative 15.87% respective 84.13% by weight.

### Vortex

The vortex time represents an absorption speed of water-absorbent particles at 0.9 mass % of saline under stirring. Pipe 50.0±1.0 ml of 0.9% NaCl solution into a 100 mL beaker with a stir bar stirring on magnetic stirring plate at a rotation speed of 600 rpm. The liquid surface produces a vortex under stirring. Then 2.000±0.010 gram of water-absorbent particles are weighted and added into the beaker as quickly as possible, with starting the stop watch synchronously. The stopwatch is stopped when the surface of liquid becomes “still”, namely, the surface has no turbulence while the mixture may still be turning. The displayed time (in seconds) is recorded as the vortex time.

### Test Methods of Laminates

### Blood Strike Through (ST) Intake Time and Rewet

The test specimen is opened and put on the testing bench. Measure 5 mL of artificial blood solution using 5 mL syringe. Inject the artificial blood solution into the specimen as soon as possible. Once the artificial blood first contacts the specimen, simultaneously start a stopwatch and observe the artificial blood being absorbed by the specimen. Once the artificial blood enters into the absorption core, stop the stopwatch and record the first “strike-thru time” in seconds. Allow the test specimen to fully absorb the artificial blood for 5 minutes, monitored by a count-down timer. After 5 minutes, insult another 5 ml artificial blood at the center of the test specimen using the syringe. Record the second “strike-thru time” after the artificial blood is fully absorbed into the test specimen. Allow the specimen to absorb the artificial blood for 5 minutes using a count-down timer. After 5 minutes, insult the third 5 ml artificial blood at the center of the test specimen using the syringe. Record the third “strike-thru time” after the artificial blood is fully absorbed in the test specimen. Prepare 30-40 pieces of filter paper (MN 615, OD 11 cm) and record its dry weight in grams (DW₃). After 5 minutes, place the filter paper on to the test specimen at the center and place 1.2 kg circular weight with OD 10 cm (giving a pressure of 0.22 psi) on top the filter paper. Wait for 2 minutes monitored by a timer, then take out the weight and the filter paper. Weigh the filter and record its wet weight in grams (WW₃).

Rewet(g)=WW₃(g)−DW₃(g)

### Example 1

### Base Polymer Fine (Non-Surface Post-Crosslinked Fine Water-Absorbent Polymer Particles)

By continuously mixing water, 50% by weight NaOH solution and acrylic acid, a 42.7% by weight acrylic acid/sodium acrylate solution was prepared such that the degree of neutralization was 69.0 mol %. After the components had been mixed, the monomer solution was cooled continuously to a temperature of 30° C. by means of a heat exchanger and degassed with nitrogen. The polyethylenically unsaturated crosslinker used was 3-tuply ethoxylated glyceryl triacrylate (purity approx. 85% by weight). The amount used, based on the acrylic acid (boaa) used, 0.35% by weight. To initiate the free-radical polymerization, the following components were used: 0.002% by weight boaa of hydrogen peroxide, metered in as a 2.5% by weight aqueous solution, 0.1% by weight boaa of sodium peroxodisulfate, metered in as a 15% by weight aqueous solution, and 0.01% by weight boaa of ascorbic acid, metered in as a 0.5% by weight aqueous solution. The throughput of the monomer solution was 40 kg/h. The individual components were metered continuously into a List ORP 10 Contikneter continuous kneader reactor (List AG, Arisdorf, Switzerland).

The reaction solution had a feed temperature of 30° C. The residence time of the reaction mixture in the reactor was approx. 15 minutes.

Some of the polymer gel thus obtained was extruded with an SLRE 75R extruder (Sela Maschinen GmbH; Harbke; Germany). The temperature of the polymer gel in the course of extrusion was 85° C. The perforated plate had 12 holes having a hole diameter of 8 mm. The thickness of the perforated plate was 16 mm. The ratio of internal length to internal diameter of the extruder (L/D) was 4. The specific mechanical energy (SME) of the extrusion was 26 kWh/t. The extruded polymer gel was distributed on metal sheets and dried at 175° C. in an air circulation drying cabinet for 90 minutes. The loading of the metal sheets with polymer gel was 0.81 g/cm².

The dried polymer gel was ground by means of a one-stage roll mill (three milling runs. 1st milling run with gap width 1000 μm, 2nd milling run with gap width 600 μm and 3rd milling run with gap width 400 μm). The ground dried polymer gel with a particle size less than 150 was collected as base polymer fine (Non-surface post-crosslinked fine water-absorbent polymer particles). The property characterization was shown in table 2.

### Example 2

### Surface Crosslinking Process

1.2 kg of base polymer fine from example 2 was coated in Pflugschar M5 plowshare mixer with heating jacket (Gebr. Loedige Maschinenbau GmbH; Paderborn, Germany) at 23° C. and a shaft speed of 200 revolutions per minute by means of a two-substance spray nozzle with 54.6 g of a mixture of 0.07% by weight of N-hydroxyethyl-2-oxazolidinone, 0.07% by weight of 1,3-propanediol, 0.7% by weight of propylene glycol, 2.27% by weight of a 22% by weight aqueous aluminum lactate solution, 0.448% by weight of a 0.9% by weight aqueous sorbitan monolaurate solution and 0.992% by weight of isopropanol, the percentages by weight each being based on base polymer fine from example 1.

After the spray application, the product temperature was increased to 185° C. and the reaction mixture was held at this temperature and a shaft speed of 50 revolutions per minute for 35 minutes. The resulting product was cooled to ambient temperature and the fraction with a particle size of less than 150 μm was collected as surface crosslinked (SXL) polymer fine (surface post-crosslinked fine water-absorbent polymer particles). The property characterization was shown in table 2.

### Example 3

The base polymer fine from example 1 and the SXL polymer fine from example 2 were blended with a weight ratio of 3:1 to obtain a mixture polymer fine as example 3. The property characterization was shown in table 2.

TABLE 2
Property characterization of polymer fines

| | | 45-150 | >200 | | | 0.3 psi |
|---|---|---|---|---|---|---|
| | <45 | μm | μm | Vortex | CRC | AAP |
| Exp. | μm [%] | [%] | [%] | [s] | [g/g] | [g/g] |
| Example 1 | 23.3 | 71.5 | 4.3 | 14.8 | 32.4 | 7.3 |
| Example 2 | 21.1 | 70.0 | 8.7 | 21.8 | 11.5 | 15.7 |
| Example 3 | | | | 18.2 | 26.8 | 10.0 |

### Example 4

### Agglomeration Process

600 g of the polymer fines from example 3 was heated to 50° C. in the oven, then was put into a 50° C. preheated Pflugschar M5 plowshare mixer (Gebr. Loedige Maschinenbau GmbH; Paderborn, Germany) with maximum rotation speed of 370 rpm. 235 g of D.I water, 2.5 g of Sokalan® CP45 (BASF SE, Ludwigshafen, Germany), 62 g of isopropanol and 1 g of Denacol® 810 (Ethylene glycol diglycidyl ether, Nagase Chemicals, Ltd., Osaka, Japan) were mixed as the binder solution and sprayed to the example 4 with a spraying speed of 45 ml/min, thereafter the mixture was stirred at a high speed for 3 minutes. Then the mixer was stopped and the obtained hydrogel was dried at 130° C. for 1.5 hours, pulverized by a blender (Oster, United States), and finally sized to 106-850 μm. The property characterization was shown in table 3.

### Example 5

600 g of the polymer fines from example 3 was heated to 50° C. in the oven, then was put into a 50° C. preheated Pflugschar M5 plowshare mixer (Gebr. Loedige Maschinenbau GmbH; Paderborn, Germany) with maximum rotation speed of 400 rpm. 150 g of D.I water, 9.5 g of Lupasol® PN80 (BASF SE, Ludwigshafen, Germany), 93.6 g of isopropanol, 3 g of Sipernat®22S (Evonik Industries AG, Essen, Germany) and 1 g of Denacol® 810 (Ethylene glycol diglycidyl ether, Nagase Chemicals, Ltd., Osaka, Japan) were mixed as the binder solution and sprayed to the example 4 with a spraying speed of 45 ml/min, thereafter the mixture was stirred at a high speed for 3 minutes. Then the mixer was stopped and the obtained hydrogel was dried at 130° C. for 1.5 hours, pulverized by a blender (Oster, United States), and finally sized to 106-850 μm. The property characterization is compared to reference samples from Sumitomo Seika Chemicals Co., Ltd. Japan. (Aquakeep SA 60N) and Formosa Plastic Group, China (NB283 FHW) as shown in the table 3.

### Example 6

The procedure was as example 5, except that 2.4 g Lupasol® PN80 were used for agglomeration. The property characterization was shown in table 3.

TABLE 3
Property characterization of SAPs

| | | | Bulk | PSD % | |
|---|---|---|---|---|---|
| | CRC | Vortex | density | <150 | >850 |
| | g/g | Sec | g/ml | μm | μm |
| Example 4 | 18.9 | 5 | 0.45 | 34.4 | 6.6 |
| Example 5 | 22.4 | 7 | 0.42 | 2.0 | 15.6 |
| Example 6 | 21.1 | 7 | 0.46 | 27.2 | 10.7 |
| Aquakeep SA | 34.0 | 29 | 0.70 | | |
| 60N, Sumitomo | | | | | |
| Seika Chemicals | | | | | |
| Co., Ltd. Japan | | | | | |
| NB283 FHW, | 31.6 | 46 | 0.63 | | |
| Formosa Plastic | | | | | |
| Group, China | | | | | |

### Example 7

### Blood Absorption Time

The blood absorption time was determined by the time of 1 g super absorbent polymer absorbing 5 ml artificial blood (as described above). Weight 1.000±0.005 g super absorbent polymer into a 100 mL beaker, then a 5.0 ml artificial blood (23±1° C.) is poured into the beaker as quickly as possible, with starting the stop watch synchronously. The stopwatch was stopped when there was no fluid in the beaker, namely, all the liquid was absorbed by the super absorbent polymer. The displayed time (in seconds) was recorded as the blood absorption time and the results were shown in the table 4.

The result show that the example 4 and example 5 show a much faster blood acquisition time compared with the reference samples Aquakeep SA60N and NB283 FHW.

### Example 8

### Milk Absorption Time

The milk absorption time was determined by the time of 1 g super absorbent polymer absorbing 10 ml formulated milk. The formulated milk was prepared by weighting 10.000±0.005 g formulated milk powder (Aptamil Pre, Germany) and dissolved it into 60.0 ml deionized water. Weight 1.000±0.005 g super absorbent polymer into a 50 mL beaker, then 10.0 ml formulated milk (23±1° C.) was poured into the beaker as quickly as possible, with starting the stop watch synchronously. The stopwatch was stopped when there was no fluid in the beaker, namely, all the liquid was absorbed by the super absorbent polymer. The displayed time (in seconds) was recorded as the milk absorption time and the results were shown in the table 4.

The result show that the example 4 and example 5 show a much faster formulated milk absorption time compared to Aquakeep SA60N and NB283 FHW

TABLE 4
Artificial blood acquisition time and
formulated milk acquisition time

| | Artificial blood | Formulated milk |
|---|---|---|
| | acquisition | acquisition |
| Sample | time/sec | time/sec |
| Aquakeep | 99 | 30 |
| SA60N | | |
| NB283 FHW | 44 | 18 |
| Example 4 | 21 | 7 |
| Example 5 | 25 | 11 |

### Example 9

### Handmade Fluid-Absorbent Articles

Laminates (absorbent core) were prepared by distributing 80 g per square meter (gsm) water-absorbent polymer and 320 gsm small fluff with a fiber length of 2 to 3 mm, a density of 0.6±0.05 g/cm₃ (pH 6-8) on to a 40 gsm tissue paper, roll, air-thru bonded and a width of 17 cm evenly. The laminates were compressed by passing through a compression rolls to make surface smooth. As compression roller, hand compression roller from Taobao, maximum pressure 15KN was used. Normally, the structure consists of five layers of superabsorbent and six layers of small fluff that each 40 cm long and 10 cm width, with a total laminate core length of 40 cm and width of 10 cm.

### Example 10

Different laminates were produced according to example 9 with water-absorbent polymers shown in table 5.

TABLE 5
Absorption articles with different SAPs

| Laminate | Water-absorbent polymer |
|---|---|
| 1 | Example 4 |
| 2 | Aquakeep SA60N, Sumitomo |
| | Seika Chemicals Co., Ltd. Japan |

### Example 11

For the laminates as descripted in example 10 the Blood Strike Through (ST) intake time and Rewet is determined. The results are summarized in Table 6.

TABLE 5
Blood strike through (ST)
time and Rewet results

| | | ST | ST | ST | |
|---|---|---|---|---|---|
| laminate | SAP | 1^{st}/sec | 2^{nd}/sec | 3^{rd}/sec | Rewet/g |
| 2 | Aquakeep SA60N | 29 | 271 | 858 | 2.0 |
| 1 | Example 4 | 10 | 102 | 421 | 2.4 |

The results show that the laminate with example 4 as the superabsorbent shows a much faster Blood Strike Through (ST) intake time but a comparable low Rewet compared to the reference sample Aquakeep SA60N.

1. A feminine hygiene absorbent article, comprising
(A) an upper liquid-pervious layer,
(B) a lower liquid-impervious layer,
(C) a fluid-absorbent core between the layer (A) and the layer (B), comprising 5% to 50% by weight of water-absorbent polymer particles G and not more than 95% by weight of fibrous material, based on the sum of water-absorbent polymer particles G and fibrous material;
(D) an optional acquisition-distribution layer between (A) and (C),
(E) an optional tissue layer disposed immediately above and/or below (C); and
(F) other optional components,
wherein the water-absorbent polymer particles G are obtainable by agglomerating a blend of non-surface post-crosslinked fine water-absorbent polymer particles and surface post-crosslinked fine water-absorbent polymer particles, and drying, grinding, sieving and classifying the agglomerated water-absorbent polymer particles.

2. The feminine hygiene absorbent article according to claim 1, wherein the ratio of the non-surface post-crosslinked fine water-absorbent polymer particles to the surface post-crosslinked fine water-absorbent polymer particles is at least 2 to 1 by weight.

3. The feminine hygiene absorbent article according to claim 1, wherein the ratio of the non-surface post-crosslinked fine water-absorbent polymer particles to the surface post-crosslinked fine water-absorbent polymer particles is at least 3 to 1 by weight.

4. The feminine hygiene absorbent article according to claim 1, wherein the water-absorbent polymer particles G have a blood acquisition time of less than 30 s, wherein the blood acquisition time is measured according to the Blood Acquisition Time test method.

5. The feminine hygiene absorbent article according to claim 1, wherein the water-absorbent polymer particles G have a milk absorption time of less than 15 s, wherein the milk absorption time is measured according to the Milk Absorption Time test method.

6. The feminine hygiene absorbent article according to claim 1, wherein the fine water-absorbing polymer particles are agglomerated to obtain water-absorbent polymer particles G by using a solution or suspension comprising,
a) 0.04 to 1.2% by weight water-soluble or water-dispersible polymeric binders or a mixture thereof, based on the water-absorbent polymer particles,
b) 20 to 70% by weight of water based on the water-absorbent polymer particles, and
c) 5 to 20% by weight of a water-miscible organic solvent based on the water-absorbent polymer particles.

7. The feminine hygiene absorbent article according to claim 6, wherein the water-soluble polymeric binders comprise carboxymethyl cellulose, starch, dextran, polyvinylamine, polyethyleneimine, polyvinylalcohol, polyacrylic acid and its salts, polyethylene oxide, polyethyleneglycol and/or chitosan.

8. The feminine hygiene absorbent article according to claim 6, wherein the water-dispersible polymeric binders comprise homo- and copolymers of vinyl esters, vinyl acetate homopolymers and vinyl acetate copolymers with ethylene, acrylates, maleic acid esters, vinyl-amides, other vinylacyl derivatives, homo- and co-polymers of acrylic and methacrylic acid esters, copolymers of methyl methacrylate, n-butyl acrylate, and/or 2-ethylhexyl acrylate.

9. The feminine hygiene absorbent article according to claim 6, wherein the water-miscible organic solvents comprise alcohols, tetrahydrofuran and/or acetone.

10. The feminine hygiene absorbent article according to claim 1, wherein the fluid absorbent core (C) comprises at maximum 25% by weight of water-absorbent polymer particles G and not less than 75% by weight of fibrous material, based on the sum of water-absorbent polymer particles and fibrous material.

11. The feminine hygiene absorbent article according to claim 1, wherein the fluid-absorbent core (C) comprises at least two layers each comprising 5% to 25% by weight of water-absorbent polymer particles G and 75% to 95% by weight of fibrous material, based on the sum of water-absorbent polymer particles and fibrous material.
